(19) United States
(12) Patent Application Publication
(10) Pub. No.: US 2024/0257910 A1
(43) Pub. Date: Aug. 1, 2024

(54) METHOD AND ELECTRONIC DEVICE FOR PREDICTING PATCH-LEVEL GENE EXPRESSION FROM HISTOLOGY IMAGE BY USING ARTIFICIAL INTELLIGENCE MODEL

(71) Applicant: RESEARCH & BUSINESS FOUNDATION SUNGKYUNKWAN UNIVERSITY, Suwon-si (KR)

(72) Inventors: Young Min CHUNG, Gyeonggi-do (KR); Kyeong Chan IM, Gyeonggi-do (KR); Joo Sang LEE, Seoul (KR)

(73) Assignee: RESEARCH & BUSINESS FOUNDATION SUNGKYUNKWAN UNIVERSITY, Suwon-si (KR)

(21) Appl. No.: 18/425,291

(22) Filed: Jan. 29, 2024

(30) Foreign Application Priority Data
Jan. 30, 2023 (KR) 10-2023-0011857
Oct. 13, 2023 (KR) 10-2023-0136981

(51) Int. Cl. G16B 25/10; G16B 40/10

(52) U.S. Cl. CPC G16B 25/10; G16B 40/10

(57) ABSTRACT

A method, performed by an electronic device, of predicting gene expression from a histology image by using an artificial intelligence model may include identifying a first patch of the histology image divided into a plurality of patches, initial feature data of the first patch, and initial feature data of a second patch of the histology image, extracting global feature data of the first patch based on the initial feature data of the first patch and the initial feature data of the second patch, by using a first artificial intelligence model, extracting local feature data of the first patch from the first patch by using a second artificial intelligence model, and predicting a gene expression value for the first patch based on the global feature data of the first patch and the local feature data of the first patch, by using a third artificial intelligence model.

### CROSS-REFERENCE TO RELATED APPLICATION

This application is based on and claims priority under 35 U.S.C. § 119 to Korean Patent Application No. 10-2023-0011857, filed on Jan. 30, 2023, in the Korean Intellectual Property Office, and Korean Patent Application No. 10-2023-0136981, filed on Oct. 13, 2023, in the Korean Intellectual Property Office, the disclosures of which are incorporated by reference herein in their entirety.

### BACKGROUND

### 1. Field

The disclosure relates to a method and electronic device for predicting patch-level gene expression from a histology image by using an artificial intelligence model.

This research was supported by the Samsung Future Technology Promotion Project [SRFC-MA2102-05].

### 2. Description of the Related Art

Functions of many biological systems, such as embryos, brains, or tumors, may rely on the spatial architecture of cells in tissue and spatially coordinated regulation of genes of the biological systems. In particular, cancer cells may show significantly different coordination of gene expression from their healthy counterparts. Thus, a deeper understanding of distinct spatial organization of the cancer cells may lead to a more accurate diagnosis and treatment for cancer patients.

The recent development of the large-scale spatial transcriptome (ST) sequencing technology enables quantification of messenger ribonucleic acid (mRNA) expression of a large number of genes within a spatial context of tissues and cells along a predefined grid in a histology image. However, advanced ST sequencing technology may incur high costs.

### SUMMARY

Additional aspects will be set forth in part in the description which follows and, in part, will be apparent from the description, or may be learned by practice of the presented embodiments of the disclosure.

The present disclosure may be implemented in various ways, including a method, a system, a device, or a computer program stored in a computer-readable storage medium.

According to an embodiment, a method, performed by an electronic device, of predicting gene expression from a histology image by using an artificial intelligence model may include identifying a first patch of the histology image divided into a plurality of patches, initial feature data of the first patch, and initial feature data of a second patch of the histology image. In an embodiment, the method may include extracting global feature data of the first patch based on the initial feature data of the first patch and the initial feature data of the second patch, by using a first artificial intelligence model. In an embodiment, the method may include extracting local feature data of the first patch from the first patch by using a second artificial intelligence model. In an embodiment, the method may include predicting a gene expression value for the first patch based on the global feature data of the first patch and the local feature data of the first patch, by using a third artificial intelligence model.

A program for executing, on a computer, a method of predicting patch-level gene expression from a histology image by using an artificial intelligence model according to an embodiment may be recorded on a computer-readable recording medium.

According to an embodiment, an electronic device for predicting patch-level gene expression from a histology image by using an artificial intelligence model may include a memory storing one or more instructions, and at least one processor configured to execute the one or more instructions stored in the memory. In an embodiment, the at least one processor may be configured to identify a first patch of the histology image divided into a plurality of patches, initial feature data of the first patch, and initial feature data of a second patch of the histology image. In an embodiment, the at least one processor may extract global feature data of the first patch based on the initial feature data of the first patch and the initial feature data of the second patch, by using a first artificial intelligence model. In an embodiment, the at least one processor may extract local feature data of the first patch from the first patch by using a second artificial intelligence model. In an embodiment, the at least one processor may predict a gene expression value for the first patch based on the global feature data of the first patch and the local feature data of the first patch, by using a third artificial intelligence model.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A to 1C are diagrams illustrating examples of predicting patch-level gene expression from a histology image, according to an embodiment;

FIG. 2 is a flowchart of a method, performed by an electronic device, of predicting patch-level gene expression from a histology image by using an artificial intelligence model, according to an embodiment;

FIG. 3 is a diagram illustrating an example of a model configured to predict patch-level gene expression from a histology image, according to an embodiment;

FIG. 4 is a diagram illustrating an example of encoding positional information of a patch in a histology image, according to an embodiment;

FIG. 5 is a diagram illustrating an example of encoding positional information of a patch in a histology image, according to an embodiment;

FIG. 6 is a diagram illustrating an example of a result of patch-level GNAS gene expression prediction from a histology image, and GNAS gene expression ground truth, according to an embodiment;

FIGS. 7A to 7F are diagrams quantitatively showing the performance of artificial intelligence models to predict gene expression, according to an embodiment;

FIGS. 8A and 8B are diagrams qualitatively showing the prediction performance of an artificial intelligence model according to an embodiment;

FIGS. 9A and 9B are diagrams showing a performance contribution of each component of an artificial intelligence model according to an embodiment; and

FIG. 10 is a block diagram illustrating an example of an electronic device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As the disclosure allows for various changes and numerous embodiments, particular embodiments will be illustrated in the drawings and described in detail. However, this is not intended to limit the disclosure to particular modes of practice, and it is to be appreciated that all changes, equivalents, and substitutes that do not depart from the spirit and technical scope of the disclosure are encompassed in the disclosure.

In describing embodiments, detailed descriptions of the related art will be omitted when it is deemed that they may unnecessarily obscure the gist of the disclosure. In addition, ordinal numerals (e.g., ‘first’ or ‘second’) used in the description of an embodiment are identifier codes for distinguishing one component from another.

Hereinafter, embodiments of the disclosure will be described in detail with reference to the accompanying drawings to allow those of skill in the art to easily carry out the embodiments. The disclosure may, however, be embodied in many different forms and should not be construed as being limited to the embodiments set forth herein. Prior to the detailed description of the disclosure, the terms used herein may be defined or understood as follows.

In the present specification, it should be understood that when components are “connected” or “coupled” to each other, the components may be directly connected or coupled to each other, but may alternatively be connected or coupled to each other with a component therebetween, unless specified otherwise. In addition, ‘connection’ may refer to a wireless connection or a wired connection.

Also, as used herein, a component expressed as, for example, ‘ . . . er (or)’, ‘ . . . unit’, ‘ . . . module’, or the like, may denote a unit in which two or more components are combined into one component or one component is divided into two or more components according to its function. In addition, each component to be described below may additionally perform, in addition to its primary function, some or all of functions of other components take charge of, and some functions among primary functions of the respective components may be exclusively performed by other components.

In the disclosure, a ‘histology image’ may refer to a digital image generated by photographing, with a slide scanner, a microscope, a camera, or the like, a slide that has been fixed and stained (e.g., hematoxylin and eosin (H&E)-stained) through a series of chemical processes for observing tissue or the like removed from the human body. For example, a ‘histology image’ may refer to a digital image captured by using a microscope and may include information about cells, tissues, and/or structures in the human body. In an embodiment, a ‘histology image’ may refer to a whole slide image (WSI) including a high-resolution image of a whole slide, or a pathology slide image obtained by photographing a pathology slide. In an embodiment, a ‘histology image’ may refer to a part of a high-resolution WSI. A ‘histology image’ may include one or more patches.

In the present specification, the terms ‘patch’, ‘patch image’, ‘spot’, and ‘spot image’ may be used interchangeably to refer to a partial region of a histology image. For example, a ‘patch’, a ‘patch image’, a ‘spot’, and a ‘spot image’ may include at least some of a plurality of pixels of a histology image. In an embodiment, a histology image may be divided into a plurality of quadrangular patches, but the shape of a patch is not limited to a quadrangular shape. For example, the shape of a patch may vary depending on the shape of an effective region (e.g., a tissue region or a cell region) included in the histology image. In an embodiment, a histology image may be divided into a plurality of patches that do not overlap each other, but the disclosure is not limited thereto. For example, a histology image may be divided such that at least a partial region of a patch overlaps at least a partial region of another patch.

In the present specification, ‘gene expression’ may refer to information about expression of one or more genes. For example, information about expression of one or more genes may include whether the one or more genes are expressed and an expression degree, an expression level, an expression value (e.g., a messenger ribonucleic acid (mRNA) expression value), an expression pattern, and the like of the one or more genes. Thus, ‘predicting gene expression’ may refer to predicting information about expression of one or more genes to be predicted. The one or more genes to be predicted may be genes expressed in cells or tissues of organisms, such as A2M, AKAP13, BOLA3, CDK6, C5orf38, DHX16, FAIM2, HSPB11, MED13L, MID1IP1, or ZFP36L2, but are not limited thereto. In an embodiment, genes to be predicted may be different from each other depending on cells and/or tissues. For example, genes of which expression in tissue of a first region of an organism is to be predicted and genes of which expression in tissue of a second region of the organism is to be predicted may be the same as or different from each other, or some of them are different from each other.

In the present specification, ‘patch-level gene expression’ may refer to gene expression for each patch. In the present specification, ‘gene expression for a patch’, ‘gene expression of a patch’, and ‘gene expression information about a patch’ may refer to gene expression at the position of a particular patch in a histology image. For example, ‘gene expression for a patch’, ‘patch-level gene expression’, and ‘gene expression information about a patch’ may include whether one or more genes are expressed at the position of a particular patch in a histology image, and an expression degree, an expression level, an expression value, and the like of the one or more genes.

In the present specification, ‘local information’ may refer to information and data limited to an arbitrary patch in a histology image. In an embodiment, ‘local information’ of a particular patch may include feature data derived from the particular patch. In an embodiment, ‘local information’ may include information and data of which the meaning is recognizable. In an embodiment, ‘local information’ may include information and data of which the direct meaning is not recognizable. For example, ‘local information’ of a particular patch may include feature data output by inputting the particular patch (or data about the particular patch) into an artificial intelligence model configured to extract features from input data (e.g., image data).

In the present specification, ‘global context’ and ‘global information’ may refer to overall and global information and data of patches in a histology image. That is, unlike the local information, ‘global context’ and ‘global information’ may not be limited to information and data of an arbitrary patch. In an embodiment, ‘global information’ of a particular patch may include feature data derived based on not only the particular patch but also other patches. For example, the ‘global information’ of a particular patch may include feature data derived considering correlations between data regarding the particular patch and data regarding other patches, and/or the position of the particular patch in a histology image. In an embodiment, ‘global context’ and ‘global information’ may include information and data of which the meaning is recognizable. In an embodiment, ‘global context’ and ‘global information’ may include information and data of which the direct meaning is not recognizable. For example, ‘global information’ (or ‘global context’) of a particular patch may include feature data output by inputting a plurality of patches (or data regarding the plurality of patches) into an artificial intelligence model configured to extract features from input data (e.g., image data, vector data, or feature map data).

In the present specification, that an arbitrary component, an arbitrary model, or an arbitrary module performs an arbitrary operation may mean that an electronic device (or a processor of the electronic device) including the component, model, or module performs/executes the operation. In an embodiment, the electronic device may perform/execute an operation, instruction, or calculation by the component, model, or module. In an embodiment, the electronic device may perform an operation by using the component, model, or module.

In the present specification, ‘extracting, calculating, predicting, or generating C by using model A (or based on model A), based on B (or from B)’ may mean obtaining, generating, or identifying C by inputting at least one of B or data associated with B into model A such that at least one of C or data associated with C is output from model A, but the disclosure is not limited thereto. For example, the data associated with B may refer to at least one of data generated by performing an arbitrary process (e.g., preprocessing), operation, or computation on B, data extracted or calculated from B, data calculated, generated, or determined based on B, and part of B. For example, the data associated with C may refer to at least one of data from which C is generated by performing an arbitrary process (e.g., postprocessing), operation, or computation, data from which C is extracted or calculated, data that is the basis for calculating, generating, or determining C, and data including at least part of C. In addition, in ‘extracting, calculating, predicting, or generating C by using model A (or based on model A), based on B (or from B)’, other additional data may be input into model A in addition to at least one of B or data associated with B that is input into model A, and similarly, model A may also output other additional data in addition to at least one of C or data associated with C that is output by model A.

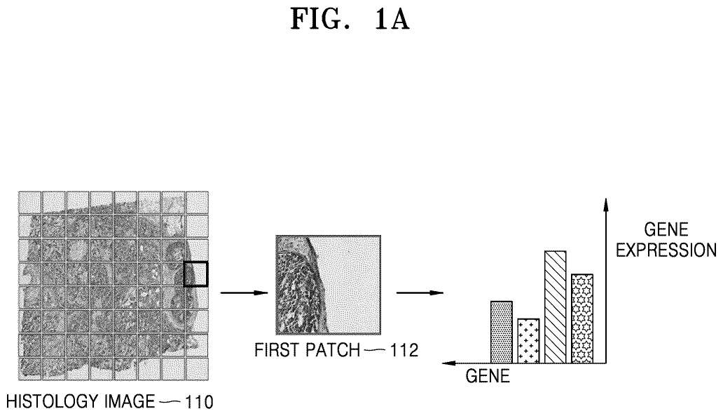

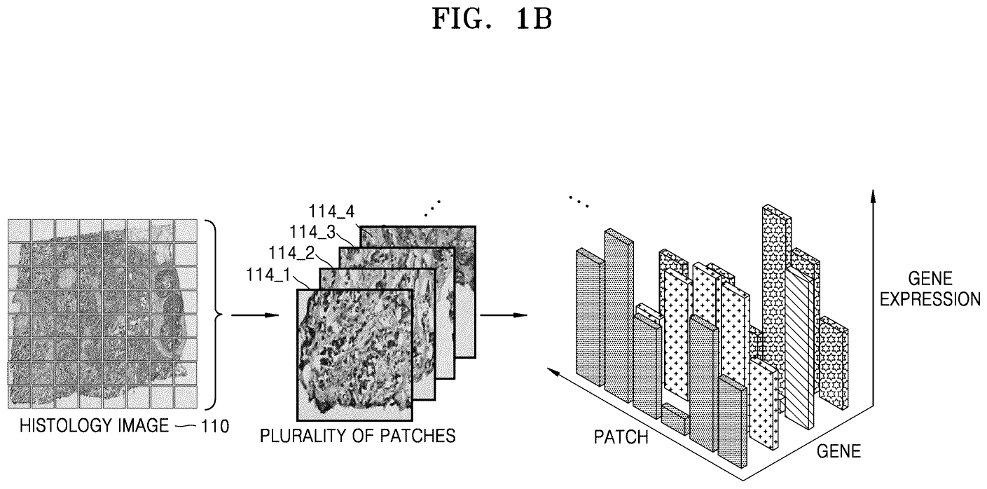

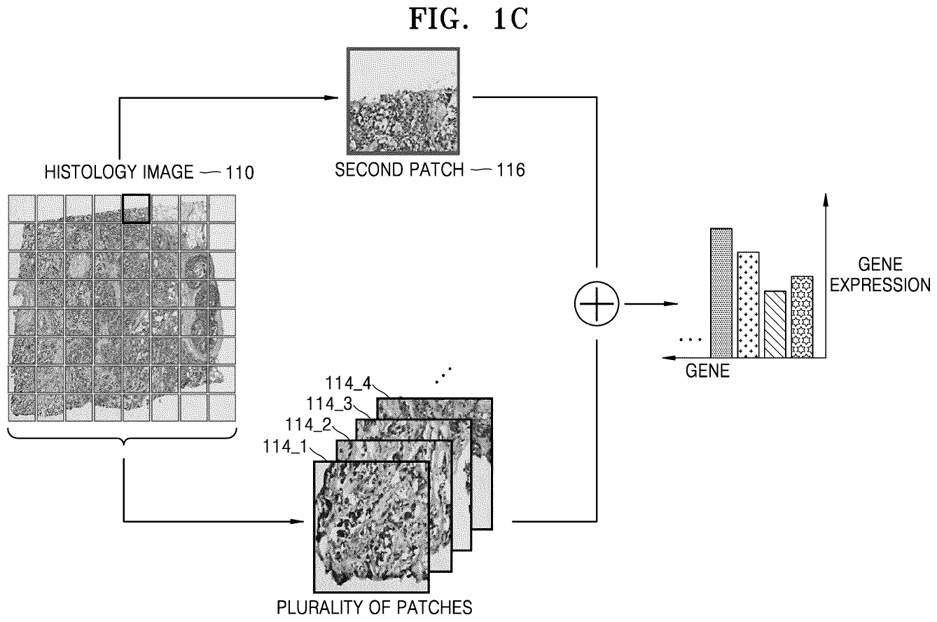

FIGS. 1A to 1C are diagrams illustrating examples of predicting patch-level gene expression from a histology image, according to an embodiment.

FIG. 1A is a diagram illustrating an example of predicting patch-level gene expression from a histology image 110, based on local information of patches of the histology image 110. In an embodiment, an electronic device (e.g., at least one processor of the electronic device) may predict gene expression for a particular patch by using only data of the particular patch (i.e., a particular spot image) in the histology image 110. That is, the electronic device may predict gene expression for a particular patch by using only local information limited to the particular patch in the entirety of the histology image 110. Referring to FIG. 1A, the electronic device may predict gene expression in a first patch 112 based on only the first patch 112 among a plurality of patches of the histology image 110. In this case, the electronic device may predict gene expression for the first patch 112 by using only information and data included in the first patch 112, without considering other patches or overall information of the histology image 110.

In an embodiment, the electronic device may predict spatial gene expression patterns in individual patches of the histology image 110 (or a WSI) by using a pre-trained convolutional neural network (CNN)-based model. For example, the electronic device may predict a gene expression value for each patch by dividing the histology image 110 into a plurality of patches and inputting each of the patches as one input into an artificial intelligence model. For example, the artificial intelligence model operating in the electronic device may receive each patch as individual input data and output a gene expression prediction result for the patch. That is, the first patch 112 may not affect gene expression prediction results for other patches, and similarly, a gene expression prediction result for the first patch 112 may not be affected by the other patches.

FIG. 1B is a diagram illustrating an example of predicting patch-level gene expression from a histology image 110, based on global information of patches of the histology image 110. In an embodiment, the electronic device may predict gene expression for a plurality of patches 114_1, 114_2, 114_3, 114_4, . . . , by using data of the plurality of patches 114_1, 114_2, 114_3, 114_4, . . . (e.g., all patches) in the histology image 110 together. That is, the electronic device may predict gene expression for the patches based on a global context (or global information) of the patches in the histology image 110. Referring to FIG. 1B, the electronic device may predict gene expression in each patch simultaneously based on the plurality of patches 114_1, 114_2, 114_3, 114_4, . . . of the histology image 110.

In an embodiment, the electronic device may simultaneously predict spatial gene expression patterns from the plurality of patches 114_1, 114_2, 114_3, 114_4, . . . (e.g., all patches) of the histology image 110 (or a WSI) by using a vision transformer (ViT)-based model. For example, the electronic device may predict a gene expression value for each patch by dividing the histology image 110 into the plurality of patches 114_1, 114_2, 114_3, 114_4, . . . , and inputting data regarding the plurality of patches 114_1, 114_2, 114_3, 114_4, . . . as one input into an artificial intelligence model. For example, the artificial intelligence model operating in the electronic device may receive the plurality of patches 114_1, 114_2, 114_3, 114_4, . . . as input data and may output gene expression prediction results for the patches. That is, an arbitrary patch may affect gene expression prediction results for other patches.

When prediction is performed by inputting the plurality of patches 114_1, 114_2, 114_3, 114_4, . . . with large data sizes as they are, that is, inputting each patch image data as it is, into the artificial intelligence model at the same time, the computational burden on the electronic device may increase. Thus, the electronic device may extract initial features from each of the plurality of patches 114_1, 114_2, 114_3, 114_4, . . . , and use the extracted initial features as input data. For example, the electronic device may extract initial features of a first patch from the first patch and extract initial features of a second patch from the second patch. The electronic device may predict gene expression in each patch by inputting the extracted initial features of the first patch, the extracted initial features of the second patch, . . . and extracted initial features of an n-th patch as one input data into an artificial intelligence model.

In the above-described one or more embodiments, because prediction is performed by focusing on only local information or a global context, information used for the prediction may be limited. Because local properties such as the morphology of cells or cell organelles may be directly associated with gene expression, fine-grained local information may be important in gene expression prediction. In addition, because gene expression in a cell is highly correlated with gene expression in other cells in tissue, a global context may also be important in predicting gene expression. Thus, when prediction is performed by focusing on only a global context, the performance of gene expression prediction may be poor because local information of a patch is not reflected, and when prediction is performed based on only local information of a patch without considering information of other patches and overall information of an image, the performance of gene expression prediction may also be poor.

FIG. 1C is a diagram illustrating an example of predicting patch-level gene expression from the histology image 110, based on local information and global information of patches of the histology image 110. In an embodiment, in order to improve the performance of gene expression prediction, the electronic device may integrate local information with global information of patches and perform patch-level gene expression based on the integrated information. Referring to FIG. 1C, the electronic device may extract local information of a second patch 116 in the histology image 110 from the second patch 116 and extract global information of the second patch 116 based on the plurality of patches 114_1, 114_2, 114_3, 114_4, . . . of the histology image 110. The electronic device may predict gene expression for the second patch 116 by using both the local information of the second patch 116 and the global information of the second patch 116 (i.e., a global context in the histology image). That is, the electronic device may integrate the local information of the second patch 116 with the global information of the second patch 116 and predict gene expression for the second patch 116 based on the integrated information.

Furthermore, existing artificial intelligence models used to predict gene expression may not be appropriate for analysis of histology images. That is, histology images have different characteristics from generic images, and thus, models and methods used for analysis of generic images may be inappropriate for analyzing histology images. For example, an existing artificial intelligence model may be a pre-trained image network model (e.g., a CNN model) designed for analysis of generic images. For example, an existing artificial intelligence model may be a patch embedding module (or model) trained by using a small amount of tissue images accompanied with spatial transcriptome (ST) sequencing.

Due to arbitrary shapes of tissue biopsies, histology images are not easily regularized in a squared form, unlike generic images that may be used in existing image network models (e.g., ImageNet) or other general resources. It may not be easy to extract a robust representation from a high-resolution histology image with only a limited number of patches in ST data, and to properly encode the positions of the patches by using existing positional encoding methods. Therefore, it may be difficult to achieve robust prediction, that is, high prediction performance, when predicting gene expression from a histology image by using existing models and existing methods.

According to an embodiment, a method and electronic device for predicting spatial gene expression through accelerated dual embedding of a histology image (e.g., a cancer histology image) may be provided. In an embodiment, the electronic device may simultaneously embed local features and global features of a histology image and predict gene expression by using both pieces of information. For example, the electronic device may predict gene expression by using a local embedding model and a global embedding model. The local embedding model includes a pre-trained model (e.g., Resnet-18), and may learn fine-grained local features from a target patch. The global embedding model may learn global context-aware features from all patches of a histology image (e.g., a WSI) by using an artificial intelligence model (e.g., a ViT model).

In an embodiment, the electronic device may tune a model (e.g., a Resnet-18 model) pre-trained based on histology images from various sources by using a self-supervised learning scheme, to extract robust features. The electronic device may extract features (e.g., initial features) from a plurality of patches of a histology image by using the tuned model, thereby alleviating the computational cost of the global embedding model (e.g., the computational burden on the electronic device due to an operation of the global embedding model).

In an embodiment, the local embedding model may fine-tune the pre-trained model (e.g., a Resnet-18 model) to capture fine-grained local information. In an embodiment, the electronic device may perform learning by using a self-distillation scheme to improve the performance of an integrated model including the local embedding model and the global embedding model. In an embodiment, in order to overcome limitations of existing positional encoding in image analysis, the electronic device may use a positional embedding model (e.g., a positional information encoder or a positional embedding module) tailored for high-resolution histology images.

In FIGS. 1A and 1C, prediction results show gene expression values for respective genes in the form of a two-dimensional bar graph, but the method of expressing a prediction result and the form of the prediction result are not limited thereto. In addition, in FIG. 1B, a prediction result shows gene expression values for respective genes in each patch in the form of a three-dimensional bar graph, but the method of expressing a prediction result and the form of the prediction result are not limited thereto.

In addition, in FIGS. 1A to 1C, the prediction results include expression values of four types of genes, but the number of genes to be predicted is not limited thereto.

The plurality of patches 114_1, 114_2, 114_3, 114_4, . . . in FIGS. 1B and 1C may include at least one of the first patch 112 of FIG. 1A or the second patch 116 of FIG. 1C.

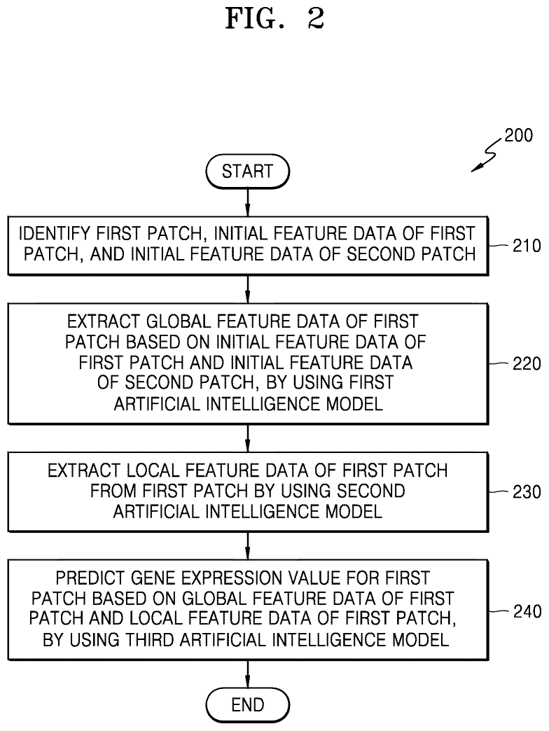

FIG. 2 is a flowchart of a method, performed by an electronic device, of predicting patch-level gene expression from a histology image by using an artificial intelligence model, according to an embodiment.

Referring to FIG. 2, a method 200 of predicting patch-level gene expression from a histology image by using an artificial intelligence model according to an embodiment may include operations 210 to 240. In an embodiment, operations 210 to 240 may be executed by at least one processor included in the electronic device. In one or more embodiments, the method 200, performed by an electronic device, of predicting patch-level gene expression from a histology image by using an artificial intelligence model is not limited to that illustrated in FIG. 2 and may further include operations not illustrated in FIG. 2 or may not include some of the operations illustrated in FIG. 2.

In operation 210, the electronic device (e.g., a processor of the electronic device) may identify a first patch of the histology image divided into a plurality of patches, initial feature data of the first patch, and initial feature data of a second patch of the histology image. For example, initial feature data of a patch may include data of at least some of pixels included in the patch. For example, initial feature data regarding a patch may be calculated by performing a feature extraction operation on data of at least some of pixels included in the patch.

In an embodiment, the initial feature data of the first patch may be extracted from the first patch by a pre-trained artificial intelligence model (e.g., a Resnet model), and the initial feature data of the second patch may be extracted from the second patch by the pre-trained artificial intelligence model. For example, the initial feature data of each patch may be vector data. For example, the pre-trained artificial intelligence model to extract initial feature data of a patch from the patch may be a lightweight model with a relatively low computational burden, a completely trained model, a feature extraction model generalized to various types of image data, a model trained by using histology images as training images, a model trained by using generic images as training images, or the like, but is not limited thereto.

In an embodiment, the electronic device may receive a histology image from another device or another server that is connected to the electronic device in a wired or wireless manner or is able to communicate with the electronic device. In an embodiment, the electronic device may generate/obtain a histology image by using an image generation device (e.g., a photographing device or an image sensor) that is embedded in, included in, or connected to the electronic device. The electronic device may divide a received, generated, or obtained histology image into a plurality of patches and extract initial feature data of a first patch from the first patch, and initial feature data of a second patch from the second patch by using the pre-trained artificial intelligence model.

In an embodiment, the electronic device may receive one or more patches of a histology image from another device or another server that is connected to the electronic device in a wired or wireless manner or is able to communicate with the electronic device. In an embodiment, the electronic device may extract initial feature data from the one or more received patches by using the pre-trained artificial intelligence model. For example, the electronic device may extract initial feature data of a received first patch from the first patch and extract initial feature data of a received second patch from the second patch. In an embodiment, the electronic device may receive initial feature data of one or more patches of a histology image from another device or another server that is connected to the electronic device in a wired or wireless manner or is able to communicate with the electronic device. For example, the electronic device may receive initial feature data of a first patch and initial feature data of a second patch of a histology image, from another device or another server.

In operation 220, the electronic device may extract global feature data of the first patch based on the initial feature data of the first patch and the initial feature data of the second patch, by using a first artificial intelligence model. In an embodiment, the first artificial intelligence model may include one or more encoders and a positional information encoder configured to encode patch positional information in a histology image. For example, the one or more encoders may perform at least one self-attention operation. In an embodiment, the electronic device may use the positional information encoder to extract the global feature data of the first patch in which positional information of the first patch is encoded. For example, the electronic device may perform at least one self-attention operation based on the initial feature data of the first patch and the initial feature data of the second patch. The electronic device may perform each of a convolution operation and a deformable convolution operation on result data of the at least one self-attention operation, based on positional information of the first patch and positional information of the second patch. The electronic device may extract the global feature data of the first patch in which the positional information of the first patch is encoded, based on result data of the convolution operation and result data of the deformable convolution operation.

In operation 230, the electronic device may extract local feature data of the first patch from the first patch by using a second artificial intelligence model. In an embodiment, the electronic device may extract first local feature data and second local feature data of the first patch from the first patch by using the second artificial intelligence model. For example, the second artificial intelligence model may include a plurality of sequentially connected layers, the first local feature data may be output data of a deepest layer (e.g., the last layer) among the plurality of layers, and the second local feature data may be data generated based on output data of another layer among the plurality of layers.

In operation 240, the electronic device may predict a gene expression value for the first patch based on the global feature data of the first patch and the local feature data of the first patch, by using a third artificial intelligence model. In an embodiment, the electronic device may generate local-global feature data of the first patch by concatenating the global feature data of the first patch with the local feature data of the first patch and predict the gene expression value for the first patch based on the generated local-global feature data of the first patch, by using the third artificial intelligence model.

In an embodiment, the first artificial intelligence model, the second artificial intelligence model, and the third artificial intelligence model used by the electronic device in operations 220 to 240 may be sub-models that constitute one artificial intelligence model. For example, the first artificial intelligence model, the second artificial intelligence model, and the third artificial intelligence model may be connected in an end-to-end manner and thus simultaneously trained/updated, but are not limited thereto. At least one of the first artificial intelligence model, the second artificial intelligence model, or the third artificial intelligence model may be individually trained/updated.

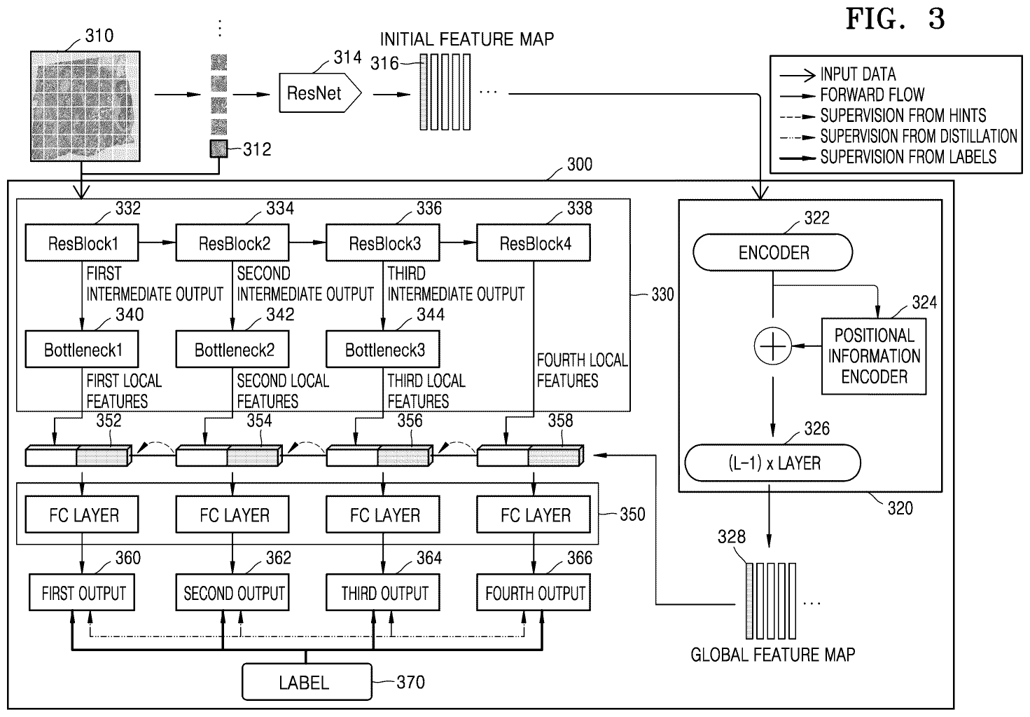

FIG. 3 is a diagram illustrating an example of a model configured to predict patch-level gene expression from a histology image, according to an embodiment.

FIG. 3 may show an example of a structure of a model 300 configured to predict patch-level gene expression from a histology image. Referring to FIG. 3, the model 300 may include a global embedding model 320, a local embedding model 330, and a combined prediction model 350 as sub-models. FIG. 3 illustrates that a model configured to extract initial feature data from a patch (e.g., a ResNet model 314) is a preprocessing model separate from the model 300, but the disclosure is not limited thereto. For example, the model configured to extract initial feature data from a patch may be included in the global embedding model 320 of the model 300.

The electronic device may identify a target patch 312 of a target histology image 310. In an embodiment, the electronic device may identify the target patch 312 by identifying the target histology image 310 and dividing the target histology image 310 into a plurality of patches. In an embodiment, the electronic device may obtain the target histology image 310 divided into the plurality of patches and identify the target patch 312 from among the plurality of patches. For example, the target histology image 310 may be input to the electronic device through an input device included in or connected to the electronic device. For example, the target histology image 310 may be received by the electronic device from another device and/or another server that is able to communicate with the electronic device in a wired or wireless manner. The patch may be an image consisting of approximately 224×224 pixels, which may be similar to the overall size of a generic image. Thus, as the number of patches increases, the amount of computation required for the electronic device to embed the patches may increase.

The electronic device may identify an initial feature map of the target histology image 310 in order to reduce the computational burden on the global embedding model 320 for a large number of (e.g., 300) patches. The initial feature map of the target histology image 310 may include initial feature data from at least some of the plurality of patches of the target histology image 310, that is, initial feature data corresponding to each of the at least some patches. In an embodiment, the electronic device may receive the initial feature map of the target histology image 310 from another device and/or server that is connected to or able to communicate with the electronic device in a wired or wireless manner. For example, the electronic device may receive initial feature data corresponding to each of at least some patches of the target histology image 310.

In an embodiment, the electronic device may extract the initial feature map based on at least some patches of the target histology image 310 by using a model configured to extract initial feature data of a patch (hereinafter, referred to as a ‘pre-model’). That is, the electronic device may perform a pre-embedding operation on at least some patches of the target histology image 310. For example, the electronic device may generate/obtain the initial feature map including initial feature data from each of at least some patches of the target histology image 310 by inputting each of the at least some patches into the pre-trained ResNet model 314. As illustrated in FIG. 3, the electronic device may generate the initial feature map including initial feature data 316 corresponding to the target patch 312 by inputting the target patch 312 into the pre-trained ResNet model 314. The pre-model (e.g., the ResNet model 314) may be a model trained to embed input patches into a d-dimensional feature vector, based on a plurality of training histology images.

The electronic device may generate a global feature map of the target histology image 310 by inputting the initial feature map of the target histology image 310 into the global embedding model 320. The global feature map of the target histology image 310 may include global feature data corresponding to at least some of the plurality of patches of the target histology image 310. For example, the global feature map of the target histology image 310 may include global feature data 328 corresponding to the target patch 312.

For example, the global embedding model 320 may include a ViT model configured to aggregate a plurality of patches. A multiple instance learning (MIL) model may be used to process a histology image (or a WSI) with a gigapixel resolution. The MIL model may use an attention-based network to aggregate patches. In an embodiment, the electronic device may use a ViT-based aggregator for considering a global context of patches.

The global embedding model 320 may encode global information from at least some patches (e.g., all patches) of the target histology image 310. In an embodiment, the electronic device may extract the global feature data 328 of the target patch 312 based on the initial feature data of the at least some patches of the target histology image 310, by using the global embedding model 320. That is, the electronic device may extract the global feature data 328 of the target patch 312 by considering not only the initial feature data of the target patch 312 but also initial feature data of other patches. The global feature data 328 of the target patch 312 may not be information limited to the target patch 312 but may be data in which global context information across the target histology image 310, such as correlations between the target patch 312 and other patches, is encoded.

The global embedding model 320 may learn correlations between at least some of the plurality of patches of the target histology image 310 considering spatial information of the at least some patches. In an embodiment, the global embedding model 320 may include one or more encoders 322 and 326 configured to encode correlations between patches. For example, the one or more encoders 322 and 326 may perform at least one self-attention operation for encoding correlations between at least some patches of the target histology image 310. For example, the global embedding model 320 may include a ViT model configured to learn long-term dependency using a self-attention operation.

In an embodiment, the global embedding model 320 may include a positional information encoder 324 for encoding spatial information of at least some patches of the target histology image 310. Thus, output data of the global embedding model 320 may include global feature data of patches in which positional information of the patches is encoded. Referring to FIG. 3, the electronic device may generate tokens for patches (e.g., first tokens corresponding to the patches) by using the first encoder 322 of the global embedding model 320 to encode correlations between the patches based on the initial feature map of the target histology image 310. The electronic device may generate tokens (e.g., second tokens corresponding to the patches) in which positional information of the patches is encoded based on the generated tokens for the patches, by using the positional information encoder 324. That is, the electronic device may extract tokens for the patches in which positional information is encoded, by inputting the initial feature map into the first encoder 322 to extract tokens for the patches, and then inputting the tokens for the patches into the positional information encoder 324. For example, one token may be vector data corresponding to one patch.

The electronic device may encode correlations between the patches L-1 times (L is a natural number greater than or equal to 1) based on output tokens of the first encoder 322 (e.g., first tokens corresponding to the patches) and output tokens of the positional information encoder 324 (e.g., second tokens corresponding to the patches), by using the other encoders 326 of the global embedding model 320. For example, the electronic device may sum up (e.g., element-wise sum) the output tokens of the first encoder 322 and the output tokens of the positional information encoder 324, input the sum to a second encoder, and sequentially perform encoding L-1 times. For example, the electronic device may sum up a first token corresponding to the target patch 312 and a second token corresponding to the target patch 312 and input the sum to the second encoder.

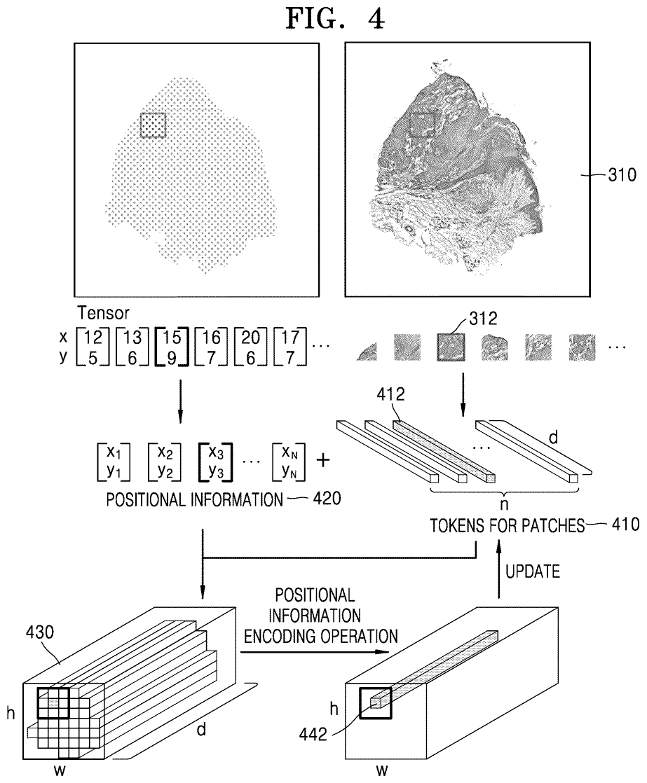

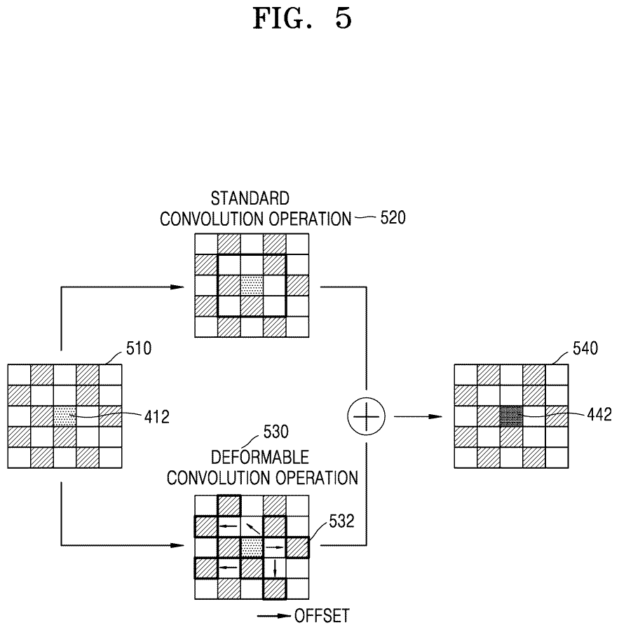

FIG. 3 illustrates that output tokens of the first encoder 322 of the global embedding model 320 are input to the positional information encoder 324, but the disclosure is not limited thereto. For example, output tokens of an e-th encoder (e is a natural number between 1 and L) of the global embedding model 320 may be input to the positional information encoder 324. In an embodiment, the electronic device may sum up output tokens of the positional information encoder 324 and output tokens of the e-th encoder, and input the sum to an (e+1)-th encoder. In an embodiment, the electronic device may calculate a global feature map by summing up the output tokens of the positional information encoder 324 and the output tokens of the e-th encoder. The positional information encoder 324 of FIG. 3 will be described below with reference to FIGS. 4 and 5, and redundant descriptions may be omitted.

The electronic device may generate/obtain local feature data of an input patch by inputting individual patches of the target histology image 310 into the local embedding model 330. For example, the local embedding model 330 may be a fine-tuned version of a pre-trained CNN model (e.g., a Resnet-18 model) to capture fine-grained local information from the input target patch 312, and predict a spatial gene expression pattern. In an embodiment, the electronic device may extract local feature data of the target patch 312 from the target patch 312 by using the local embedding model 330. Unlike the global embedding model 320, the local embedding model 330 may not operate based on other patches. That is, when extracting local feature data of the target patch 312, the electronic device may not consider information of other patches. For example, the electronic device may extract local feature data of the first patch by inputting the first patch into the local embedding model 330 and extract local feature data of the second patch by inputting the second patch into the local embedding model 330.

The electronic device may extract/calculate one or more pieces of local feature data of the target patch by inputting the target patch into the local embedding model 330. That is, the local embedding model 330 may output a plurality of pieces of output data for one input. For example, as illustrated in FIG. 3, the local embedding model 330 may output first local feature data, second local feature data, third local feature data, and fourth local feature data for the target patch 312. Each of the first local feature data, the second local feature data, and the third local feature data may be calculated based on intermediate output data generated during an inference operation of the local embedding model 330 on the target patch 312. The formats of a plurality of pieces of output data from the local embedding model 330 on the target patch 312 may be the same as each other, but are not limited thereto.

In an embodiment, the local embedding model 330 may include one or more blocks. A block may include one or more layers. For example, the local embedding model 330 may include a plurality of layers, and the plurality of layers may be divided into one or more blocks. For example, in a case in which the local embedding model 330 includes a Resnet-18 model as illustrated in FIG. 3, the Resnet-18 model may be divided into four ResBlocks and included in the local embedding model 330. In this case, the blocks of the local embedding model 330 may be connected to each other sequentially/serially.

In an embodiment, each block of the local embedding model 330 may perform an operation on the target patch 312 that is input into the local embedding model 330. For example, each block of the local embedding model 330 may calculate, generate, and output intermediate output data or deepest output data for the target patch 312. The deepest output data from the local embedding model 330 for the target patch 312 may refer to output data from a deepest block (or a deepest layer, the last block, or the last layer) of the local embedding model 330, and the intermediate output data may refer to output data from another block (or another layer) of the local embedding model 330.

Referring to FIG. 3, a first ResBlock 332 may receive the target patch 312 and perform an operation to output first intermediate output data, a second ResBlock 334 may receive the first intermediate output data and perform an operation to output second intermediate output data, a third ResBlock 336 may receive the second intermediate output data and perform an operation to output third intermediate output data, and a fourth ResBlock 338, that is, the last block, may receive the third intermediate output data and perform an operation to output deepest output data (i.e., fourth local feature data). FIG. 3 illustrates that the local embedding model 330 includes a Resnet model and each block of the local embedding model 330 is a ResBlock, but the disclosure is not limited thereto. The blocks of local embedding model 330 may include the same number or different numbers of layers.

In an embodiment, the local embedding model 330 may include one or more additional layers (e.g., bottleneck layers) configured to extract/calculate local feature data from intermediate output data. For example, referring to FIG. 3, the local embedding model 330 may include a first bottleneck layer 340 associated with the first ResBlock 332, a second bottleneck layer 342 associated with the second ResBlock 334, and a third bottleneck layer 344 associated with the third ResBlock 336. The additional layer may encode input data (i.e., intermediate output data for the target patch 312) in the same format as the deepest output data from the local embedding model 330 for the target patch 312.

The local embedding model 330 may calculate one or more pieces of local feature data based on the intermediate output data and/or the deepest output data for the target patch 312. In an embodiment, the one or more additional layers of the local embedding model 330 may extract/calculate local feature data based on intermediate output data output from the blocks associated with the one or more additional layers, respectively. For example, referring to FIG. 3, the first bottleneck layer 340 may receive the first intermediate output data for the target patch 312 from the first ResBlock 332 and output first local feature data for the target patch 312, the second bottleneck layer 342 may receive the second intermediate output data for the target patch 312 from the second ResBlock 334 and output second local feature data for the target patch 312, and the third bottleneck layer 344 may receive the third intermediate output data for the target patch 312 from the third ResBlock 336 and output third local feature data for the target patch 312. In an embodiment, the local embedding model 330 may determine the deepest output data as the fourth local feature data for the target patch 312.

The local feature data associated with the deepest block (i.e., the deepest output data) may be data better reflecting local information of the target patch 312 than local feature data associated with other blocks (i.e., local feature data from intermediate output data). For example, the fourth local feature data from the fourth block may be feature data that better reflects the local information of the target patch 312 than the second local feature data from the second block.

The electronic device may predict gene expression (e.g., a gene expression level) for the target patch 312 based on the global feature data 328 of the target patch 312 and the local feature data of the target patch 312, by using the combined prediction model 350. In an embodiment, the electronic device may combine the global feature data 328 of the target patch 312 with the one or more pieces of local feature data. For example, the electronic device may generate one or more pieces of local-global feature data of the target patch 312 by concatenating the global feature data 328 of the target patch 312 with the one or more pieces of local feature data.

The electronic device may calculate a gene expression value for the target patch 312 by inputting the local-global feature data of the target patch 312 into the combined prediction model 350. The combined prediction model 350 may include one or more predictors configured to calculate a gene expression value from local-global feature data. The predictors of the combined prediction model 350 may correspond to the blocks of the local embedding model 330, respectively.

In an embodiment, the predictor of the combined prediction model 350 may include a layer (e.g., a fully-connected (FC) layer) configured to calculate a gene expression values based on input local-global feature data. For example, the electronic device may calculate one or more gene expression values by inputting the one or more pieces of local-global feature data into the FC layers, respectively. Referring to FIG. 3, the FC layers of the combined prediction model 350 may receive first local-global feature data 352 of the target patch 312 and output a first output 360 for the target patch 312 (i.e., a first gene expression value), receive second local-global feature data 354 of the target patch 312 and output a second output 362 for the target patch 312 (i.e., a second gene expression value), receive third local-global feature data 356 of the target patch 312 and output a third output 364 for the target patch 312 (i.e., a third gene expression value), and receive fourth local-global feature data 358 of the target patch 312 and output a fourth output 366 for the target patch 312 (i.e., a fourth gene expression value). The FC layers of the combined prediction model 350 illustrated in FIG. 3 may be the same as or different from each other, or some of them may be different from each other.

The electronic device may determine a final gene expression value for the target patch 312 based on a plurality of gene expression values calculated for the target patch 312. In an embodiment, the electronic device may determine the mean of the plurality of gene expression values as the final gene expression value for the target patch 312. For example, the electronic device may determine the mean of the third output 364 and the fourth output 366 as a gene expression value for the target patch 312. For example, the electronic device may determine the mean of all gene expression values (i.e., the first output 360, the second output 362, the third output 364, and the fourth output 366) for the target patch 312, as the final gene expression value for the target patch 312. In an embodiment, the electronic device may determine any one of the plurality of gene expression values as the final gene expression value. For example, the electronic device may determine the fourth output 366 (i.e., the gene expression value associated with the deepest block) as the final gene expression value for the target patch 312.

In an embodiment, the electronic device may predict gene expression for the target patch 312 by encoding a plurality of patches of the target histology image 310 as shown in Equation (1) below by using the global embedding model 320, encoding the target patch 312 as shown in Equation (2) below by using the local embedding model 330, and combining pieces of encoding output data as shown in Equation (3) below.

$${z}_{j}^{global} = g ( {X}_{1} , {X}_{2} , … , {X}_{n} ) ∈ {ℝ}^{d} \qquad Equation ( 1 )$$

$${z}_{j}^{local} = h ( {X}_{j} ) ∈ {ℝ}^{d} \qquad Equation ( 2 )$$

$${Y}_{j} = f ( {z}_{j}^{global} ‖ {z}_{j}^{local} ) ∈ {ℝ}^{m} \qquad Equation ( 3 )$$

In Equations (1) to (3), X₁, X₂, . . . , Xₙ may denote the patches of the target histology image 310, Xⱼ∈ [symbol] may denote the target patch, n may denote the number of patches, H may denote the height of the patch, and W may denote the global width of the patch. g may denote the global embedding model 320, and zⱼ^{glodal} may denote d-dimensional global feature data corresponding to Xⱼ, that is, global feature data of Xⱼ. The global embedding model 320 in Equation (1) may include a model configured to extract initial feature data from a patch. h may denote the local embedding model 330, ans Xⱼ^{local} may denote d-dimensional local feature data of Xⱼ. In addition, ∥ may denote concatenation of vectors, f may denote the combined prediction model (i.e., a predictor), Yⱼ may denote an m-dimensional gene expression prediction result for Xⱼ, and m may denote the number of genes to be predicted.

The electronic device may perform one or more embodiments described above in an inference operation and/or a training operation of the model 300. Furthermore, the electronic device in which the inference operation of the model 300 is performed may be different from or the same as the electronic device in which the training operation of the model 300 is performed. In the inference operation of the model 300, the target histology image 310 of FIG. 3 may be an image that is a target of inference by the model 300, and the target patch 312 may be a patch that is a target of inference. In the training operation of the model 300, the target histology image 310 of FIG. 3 may be a training image for training the model 300, and the target patch 312 may be a training patch for training the model 300. Additionally or alternatively, the electronic device may perform an inference operation of the model 300 and update the model 300 at the same time.

The electronic device may train/update the model 300 by using training data. The training data may include training images (i.e., training histology images) and ground-truth labels (i.e., gene expression values) for patches of the training images. In an embodiment, the models 320, 330, and 350 constituting the model 300 may be connected to each other in an end-to-end manner, and thus trained simultaneously. The pre-model (i.e., a model configured to extract initial feature data of a patch) may not be trained together with other models. For example, the pre-model may be trained/updated separately from the models 320, 330, and 350, and may not be updated after being initially trained. For example, the pre-model and the local embedding model 330 may include models with the same structure (e.g., Resnet-18 models), but while the model 300 is being trained, parameter values of the pre-model may not be updated, and parameter values of the local embedding model 300 may be updated.

The electronic device may train the model 300 by applying a supervised learning scheme. In an embodiment, the model 300 may be trained by using a loss function based on a difference between a predicted gene expression value for the target patch 312 of the target histology image 310, and a ground-truth gene expression value (i.e., a ground-truth label). For example, the electronic device may train the model 300 such that differences between one or more gene expression values predicted by the model 300 and ground-truth gene expression values decrease. Referring to FIG. 3, parameters of the model 300 may be updated such that the difference between the first output 360 from the model 300 for the target patch 312 and a label 370, the difference between the second output 362 and the label 370, the difference between the third output 364 and the label 370, and the difference between the fourth output 366 and the label 370 decrease.

The electronic device may train the model 300 by applying a self-distillation scheme, thereby improving the performance of gene expression prediction using local-global feature data, that is, the prediction performance of the model 300. In an embodiment, the model 300 may be trained by using a loss function based on differences between gene expression values calculated based on output data from the deepest layer of the local embedding model 330 for the target patch 312 included in training images, and gene expression values calculated based on output data from other layers of the local embedding model 330 for the target patch 312.

In training the model 300, the electronic device may apply a Be Your Own Teacher (BYOT) scheme or a modification of the BYOT scheme. In this case, the prediction accuracy of the model may be improved simply by adding a small number of layers (i.e., bottleneck layers as additional layers). In an embodiment, the electronic device may perform self-distillation learning such that, among the plurality of predictors in the combined prediction model 350, the predictor associated with the deepest block of the local embedding model 330 serves as a teacher model, and the predictors associated with the other blocks of the local embedding model 330 serve as student models. The model 300 may be trained/updated by using a loss function based on differences between output data from the teacher model for the target patch 312, and output data from the student models. That is, the student models may be trained based on the output from the teacher model.

Referring to FIG. 3, the model 300 may be trained/updated such that a loss value based on the difference between the fourth output 366, which is the output from the teacher model, and the first output 360, which is the output from the student model, the difference between the fourth output 366, which is the output from the teacher model, and the second output 362, which is the output from the student model, and the difference between the fourth output 366, which is the output from the teacher model, and the third output 364, which is the output from the student model, decreases.

In an embodiment, the model 300 may be trained by using a loss function based on differences between local-global feature data generated based on output data from the deepest layer (or the deepest block) of the local embedding model 330 for the target patch 312, and local-global feature data generated based on output data from the other layers (or the other blocks) of the local embedding model 330 for the target patch 312. For example, in order to fit data (e.g., feature data or a feature map) input into the student models (i.e., student predictors) to data input into the teacher model (i.e., a teacher predictor), the model 300 may be trained such that an L2 loss between input data of the teacher model and each student model is minimized. For example, in FIG. 3, the model 300 may be trained/updated such that a loss value based on the difference between the fourth local-global feature data 358 and the third local-global feature data 356, the difference between the fourth local-global feature data 358 and the second local-global feature data 354, and the difference between the fourth local-global feature data 358 and the first local-global feature data 352 decreases.

In an embodiment, the model 300 may be trained/updated such that a difference between local-global feature data based on output data from each block of the local embedding model 330 for the target patch 312, and local-global feature data based on output data from the next block for the target patch 312 decreases. For example, as illustrated in FIG. 3, the model 300 may be trained/updated such that a loss value based on the difference between the fourth local-global feature data 358 and the third local-global feature data 356, the difference between the third local-global feature data 356 and the second local-global feature data 354, and the difference between the second local-global feature data 354 and the first local-global feature data 352 decreases.

In an embodiment, the electronic device may use loss functions of Equations (4) and (5) below to train the model 300.

$$L = {{\sum }_{i}}^{T} {L}_{i} \qquad Equation ( 4 )$$

$${L}_{i} = ( 1 - α ) \frac{1}{m} {\sum }_{k = 1}^{m} {‖ {q}_{i}^{k} - {y}^{k} ‖}_{2}^{2} + α \frac{1}{m} {\sum }_{k = 1}^{m} {‖ {q}_{i}^{k} - {q}_{T}^{k} ‖}_{2}^{2} + λ {‖ {F}_{i} - {F}_{T} ‖}_{2}^{2} \qquad Equation ( 5 )$$

In Equations (4) and (5), qᵢ^{k} may denote a predicted expression value of a k-th gene by an i-th predictor, and y^{k} may denote a ground-truth label (i.e., a ground-truth gene expression value) for the k-th gene. q_{T}^{k} may denote a predicted expression value of the k-th gene by a teacher model (i.e., a teacher predictor). Fⁱ may denote input data (e.g., an input feature map) of the i-th predictor, and F_{T} may denote input data of the teacher model. m may denote the number of genes to be predicted. α and λ may be hyperparameters for balancing between losses. Thus, according to Equation (4), the electronic device may train the model 300 by using a sum L of losses Lᵢ for the plurality of predictors of the model 300, as a loss function of the model 300.

The electronic device may train the model 300 by using the loss functions of Equations (4) and (5), but the loss function for training the model 300 are not limited to Equations (4) and (5). In an embodiment, the electronic device may train the model 300 by using loss functions including a cross-entropy loss between a predicted value and a ground-truth label, a Kullback-Leibler divergence (KLD) between the teacher model and the student models, an L2 loss (or an L1 loss) between input data or output data (e.g., feature maps) of the teacher model and the student models.

FIG. 3 illustrates an example in which the electronic device predicts gene expression for one target patch 312 among the plurality of patches of the target histology image 310, but the electronic device may predict gene expression for each of at least some patches of the target histology image 310 as a target patch. That is, the electronic device may predict a gene expression value in each of the at least some patches by inputting each of the at least some patches as a target patch into the model 300. In this case, as the target patch varies, a patch input to the local embedding model 330 may vary, and global feature data concatenated with local feature data in a global feature map of the target histology image 310 may vary.

FIG. 3 illustrates that the local embedding model 330 includes four blocks and outputs four pieces of local feature data, but the disclosure is not limited thereto. That is, the number of blocks of the local embedding model 330, and the number of pieces of output data are not limited to 4, and may be any number greater than or equal to 1.

FIG. 4 is a diagram illustrating an example of encoding positional information of a patch in a histology image, according to an embodiment.

FIG. 4 may show an example of an operation of the positional information encoder 324 of FIG. 3. The electronic device (e.g., a positional information encoder operating in the electronic device) may perform positional encoding on a histology image (or a WSI), to extract global features that reflect positional information 420 (or spatial information) of patches. In an embodiment, the electronic device may use a global embedding model including a positional information encoder, and may perform operations of encoding the positional information 420 of the patches, by using the positional information encoder. For example, the electronic device may generate tokens in which the positional information 420 is reflected, by inputting tokens 410 for the patches output from a first encoder (or an e-th encoder) of the global embedding model, into the positional information encoder.

Existing positional encoding methods that are applicable to input images having a fixed size may be difficult to be applied to input images having a variable size. The electronic device may perform positional encoding on an input image having a fixed or variable size, by using a conditional positional encoding (CPE) scheme. In an embodiment, the electronic device may restore original neighborhood positional information of the patches by reshaping data regarding the patches, and apply a convolutional layer with zero padding to the data regarding the patches in which the neighborhood positional information is restored. The electronic device may shape the data regarding the patches to which the convolutional layer is applied, and then combine the data with data regarding the original patches. In this case, even in a case in which absolute positional information of the patches is not predefined, the positional information 420 of the patches may be dynamically encoded. That is, even when the size of the input image varies, the positional information 420 of the patches may be encoded.

In an embodiment, the electronic device may generate spatial data 430 in which the positional information 420 is restored, by arranging the tokens 410 for the patches output from the first encoder (or the e-th encoder) of the global embedding model (i.e., first tokens corresponding to the patches) according to the positions of the patches within the histology image. Referring to FIG. 4, the electronic device may obtain, calculate, or generate the tokens 410 for at least some patches of the target histology image 310. The tokens 410 for the patches may be data calculated by one or more encoders of the global embedding model, and may be data reflecting correlations between the patches.

The electronic device may calculate tokens for the patches in which the positional information 420 is encoded (i.e., second tokens corresponding to the patches), by performing a positional information encoding operation on the spatial data 430 in which the positional information 420 is restored. In an embodiment, the electronic device may update the tokens 410 for the patches based on the second tokens on which the positional information encoding operation has been performed, and perform subsequent operations based on the updated tokens. For example, the electronic device may update the tokens 410 for the patches, from the existing first tokens to the second tokens, and perform the subsequent operations (e.g., an encoder operation of the global embedding model) by using the updated tokens. For example, the electronic device may update the tokens 410 for the patches with data obtained by combining (e.g., element-wise sum) the existing first tokens with the second tokens in which the positional information is encoded, and perform the subsequent operations based on the updated tokens.

Referring to FIG. 4, a token 412 for the target patch 312 (i.e., the token 412 corresponding to the target patch 312) may be calculated by the one or more encoders of the global embedding model, based on at least some patches of the target histology image 310 (e.g., initial feature data of the at least some patches). For example, the token 412 for the target patch 312 may be calculated by the one or more encoders, based on correlations between the target patch 312 and other patches. For example, the token 412 for the target patch 312 may be d-dimensional vector data, and the tokens 410 for the patches may be data in a space [symbol] (where n denotes the number of patches).

The electronic device may reshape the tokens 410 for the patches into the form of the [symbol] spatial data 430 by arranging the tokens 410 for the patches in a space [symbol] based on the positional information 420 of the patches within the target histology image 310. The electronic device may perform an operation for encoding the positional information 420, on the tokens reshaped into the form of the [symbol] spatial data 430. The electronic device may update the tokens 410 by reflecting a result of the positional information encoding operation in the existing tokens 410 for the patches. For example, the electronic device may perform a positional information encoding operation based on the token 412 for the target patch 312 and adjacent tokens to calculate a token 442 in which positional information for the target patch 312 is reflected, and update the token 412 for the target patch 312 based on the token 442 in which the position information is reflected.

In an embodiment, the electronic device may perform positional encoding in a CPE manner by using a positional encoding generator (PEG) as the positional information encoder of the global embedding model. For example, the electronic device may perform positional encoding on the patches by using a PEG that is implemented as two-dimensional convolution with a kernel k (k≥3) and (k−1)/2 zero padding. In an embodiment, the electronic device may perform positional encoding on the patches by using a pyramid PEG (PPEG) to apply PEG to WSI, as the positional information encoder of the global embedding model. For example, the electronic device may encode positional information by using a PPEG to apply kernels having various sizes and fuse them. The PPEG may apply a CPE scheme by reshaping patch tokens of a WSI into an artificial two-dimensional quadrangular feature map. The PPEG arranges patches of a WSI that is not in a quadrangular shape, in a quadrangular shape according to the order in which the patches are output from an image scanner, and thus, the patches may not be arranged according to the original positional information, and when the number of the patches is not a square number in a process of arranging the patches into a quadrangular shape, unnecessary duplication may be generated.

Meanwhile, tissue slices (i.e., histology images and WSIs) from biopsies have irregular shapes, thus, the image area of the histology images (or WSIs) may not be quadrangular unlike most generic images, and, as illustrated in FIG. 4, empty regions may exist between patches. Due to such characteristics of histology images, when a scheme that is not designed for histology images (e.g., a CPE scheme using a PEG) is applied to histology images, it may be difficult to capture the absolute positions of patches. Thus, in an embodiment, the electronic device may perform positional encoding on patches by using a position encoding generator for histology images (PEGH) as the positional information encoder of the global embedding model. The PEGH will be described below with reference to FIG. 5, and redundant descriptions may be omitted.

The positional information encoding operation of FIG. 4 may be an operation of a PEG, an operation of a PPEG, or an operation of a PEGH, but is not limited thereto.

FIG. 5 is a diagram illustrating an example of encoding positional information of a patch in a histology image, according to an embodiment.

FIG. 5 illustrates an example of the positional information encoding operation illustrated in FIG. 4, and the positional information encoding operation of FIG. 4 is not limited to that illustrated in FIG. 5. In an embodiment, the electronic device (e.g., at least one processor of the electronic device) may encode positional information by using a PEGH as a positional information encoder. For example, the electronic device may encode patch positional information by using the positional information encoder based on tokens t for patches output from an e-th encoder of the global embedding model. The electronic device may restore the original space of the patches based on positional information of the patches, and encode the positional information into the patches by using a convolutional layer.

In an embodiment, the PEGH (or the electronic device in which the PEGH operates) may generate spatial data (e.g., 430 of FIG. 4) by arranging n d-dimensional tokens [symbol] in an original space [symbol] (where h and w denote the maximum coordinate values of x and y, respectively) by using obtained relative center coordinates of the patches, and filling empty regions (e.g., regions without tissues and cells) with zeros. The electronic device may apply a convolutional layer to the generated spatial data (i.e., data in a space [symbol] where the tokens are arranged). That is, the electronic device may perform an operation by inputting the generated spatial data into a convolutional layer. As a result of performing an operation of the convolutional layer, the electronic device may obtain/generate/calculate output spatial data in the same form (e.g., [symbol] ) as input spatial data 510 of the convolutional layer.

According to an embodiment, the electronic device may perform a deformable convolution operation 530 and/or a standard convolution operation 520 by a convolutional layer of the positional information encoder PEGH. That is, the convolutional layer of the positional information encoder PEGH may include a standard convolution kernel and/or a deformable convolution kernel. Deformable convolution may be used to extend a convolutional layer of which the receptive field is limited in a rectangular grid form. The electronic device may capture arbitrary (i.e., variable) shapes of histology images by extending the receptive field of a convolution kernel into a dynamic form by using a learnable offset. In an embodiment, the electronic device may perform an operation using a deformable convolution kernel in addition to a standard convolution kernel in the same layer to process a histology image corresponding to an arbitrary shape. That is, the electronic device may perform both the standard convolution operation 520 and the deformable convolution operation 530 to extract features that better reflect the characteristics of histology images including empty spaces (i.e., spaces where no tissue appears).

In FIG. 5, at least part of [symbol] spatial data may be simplified and illustrated in an x-y plane. In an embodiment, the electronic device may perform an operation using a standard convolution kernel on tokens arranged in a space (i.e., the input spatial data 510). For example, referring to FIG. 5, the electronic device may perform the 3×3 standard convolution operation 520 based on the token 412 for the target patch arrange in the space, tokens located nearby, and empty regions (i.e., zero regions).

In an embodiment, the electronic device may perform an operation using a deformable convolution kernel on tokens arranged in a space (i.e., the input spatial data 510). Referring to FIG. 5, the electronic device may perform the 3×3 deformable convolution operation 530 based on the token 412 for the target patch arranged in the space, and valid tokens located nearby. That is, due to the offset, the electronic device may perform the deformable convolution operation 530 based on nearby valid tokens instead of zero regions. For example, in FIG. 5, due to the offset, the electronic device may perform a convolution operation based on, instead of zero data (i.e., a zero region) located to the right of the token 412 for the target patch, a token 532 located to the right of the zero data.

In an embodiment, the electronic device may generate output spatial data of the convolutional layer by combining a result of the standard convolution operation 520 with a result of the deformable convolution operation 530. For example, the electronic device may sum up the result of the standard convolution operation 520 and the result of the deformable convolution operation 530 element-wise.

In an embodiment, the electronic device may generate spatial data 540 in which positional information is encoded, by filling, with zeros, positions in the output spatial data corresponding to positions filled with zeros (i.e., zero regions) in the input spatial data 510. The spatial data 540 in which the positional information is encoded may include tokens in which positional information is encoded. For example, the spatial data in which the positional information is encoded may include a token reflecting positional information of the target patch, that is, the token 442 for the target patch in which positional information is encoded.

The electronic device may return the tokens in which the positional information is encoded, to the original form (i.e., [symbol] ) of the tokens input to the positional information encoder. The electronic device may calculate/generate global feature data (or a global feature map) in which positional information is encoded, by performing at least one self-attention operation by one or more encoders of the global embedding model based on the tokens in which positional information is encoded in the form of [symbol] . In an embodiment, the electronic device may sum up the output tokens of the positional information encoder (i.e., the tokens in which the positional information is encoded) and the input tokens of the positional information encoder element-wise, and perform at least one self-attention operation. For example, the electronic device may sum up the token 412 for the target patch that is input to the positional information encoder, and the token for the target patch that is output from the positional information encoder (i.e., the token 442 for the target patch in which the positional information is encoded) element-wise, and input the sum into the next encoder of the global embedding model.

FIG. 5 illustrates an example of performing positional encoding by using both the standard convolution operation 520 and the deformable convolution operation 530, but the disclosure is not limited thereto. For example, the electronic device may use the standard convolution operation 520 or the deformable convolution operation 530 for positional encoding. For example, the electronic device may perform positional encoding by performing another type of convolution operation for the positional encoding.

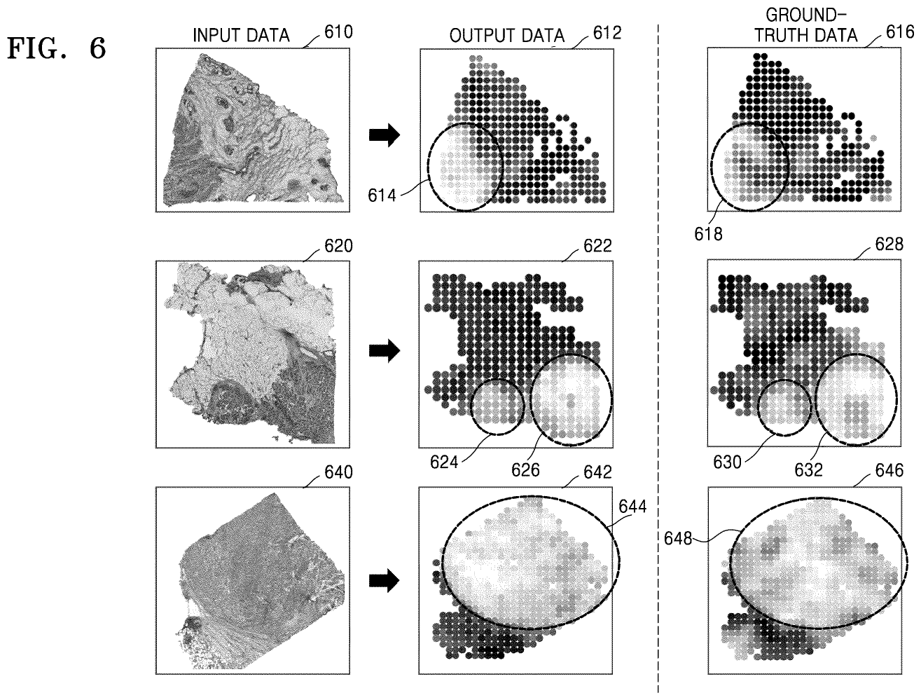

FIG. 6 is a diagram illustrating an example of a result of patch-level GNAS gene expression prediction from a histology image, and GNAS gene expression ground truth, according to an embodiment.

The electronic device may predict gene expression in each patch within an input image by applying a gene expression prediction method according to an embodiment, to the input image. In an embodiment, the electronic device may calculate an expression value of the GNAS gene in each patch of an input histology image. In an embodiment, the electronic device may determine an expression level of the GNAS gene in each patch of the input histology image. The gene expression level may be determined based on the gene expression value.

As illustrated in FIG. 6, an expression level of a particular gene may be indicated in color (e.g., brightness or saturation) in each patch region of an input image. For example, a higher brightness may indicate a lower expression level of a particular gene, and a lower brightness may indicate a higher expression level of the particular gene, but the disclosure is not limited thereto. Expression of a particular gene (i.e., an expression value or an expression level) may be indicated for each patch in various ways, such as numerical data or graph data.

As illustrated in FIG. 6, in the distribution of GNAS gene expression levels throughout the image, the first output data 612 for first input data 610 shows a similar tendency to ground-truth data 616 for the first input data 610, for example, a lower left region 614 of the first output data 612 is indicated with a high brightness like a lower left region 618 of the ground-truth data 616. Similarly, in the distribution of GNAS gene expression levels throughout the image, second output data 622 for second input data 620 shows a similar tendency to ground-truth data 628 for the second input data 620, for example, a lower middle region 624 of the second output data 622 is indicated with a high brightness like a lower middle region 630 of the ground-truth data 628, and a lower right region 626 of the second output data 622 is indicated with a high brightness to be distinguished from other regions, like a lower right region 632 of the ground-truth data 628. Similarly, in the distribution of GNAS gene expression levels throughout the image, third output data 642 for third input data 640 shows a similar tendency to ground-truth data 646 for the third input data 640, for example, an upper wide region 644 of the third output data 642 is indicated with a high brightness to be distinguished from other regions, like an upper wide region 648 of the ground-truth data 646.

The ground-truth data 616, 628, and 646 for respective input data is ground-truth data obtained through biopsies, and may represent GNAS gene expression levels in respective patches of the input images in color at the positions of the patches.

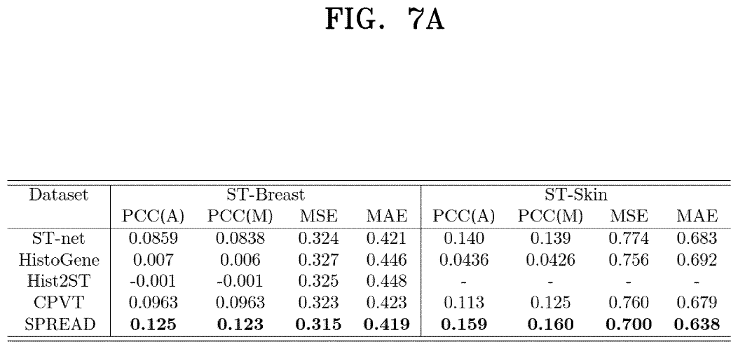

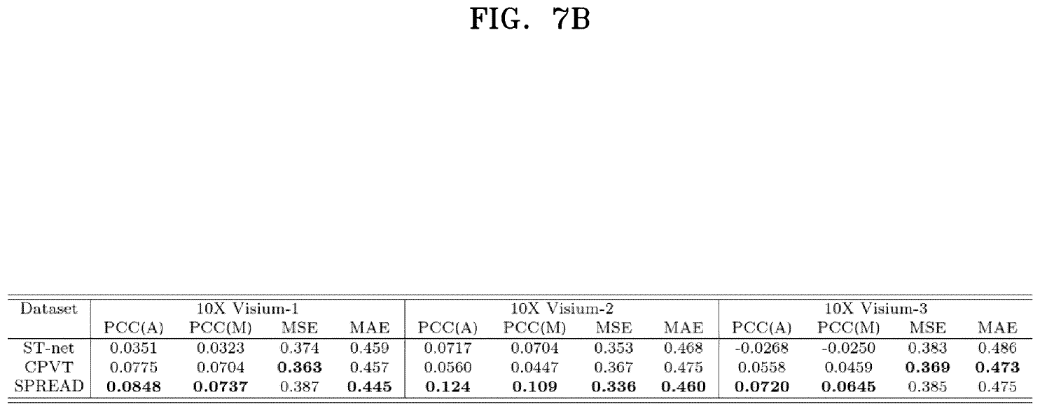

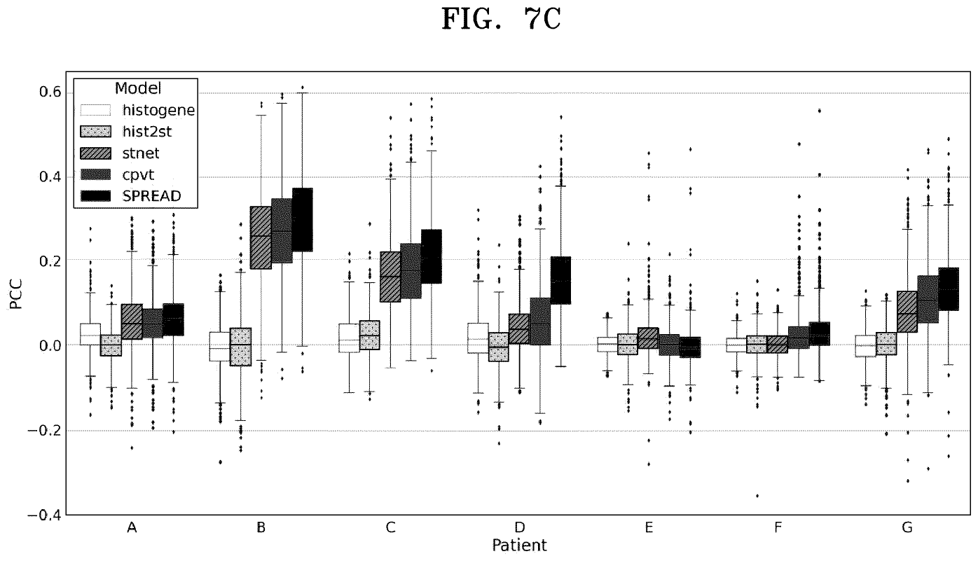

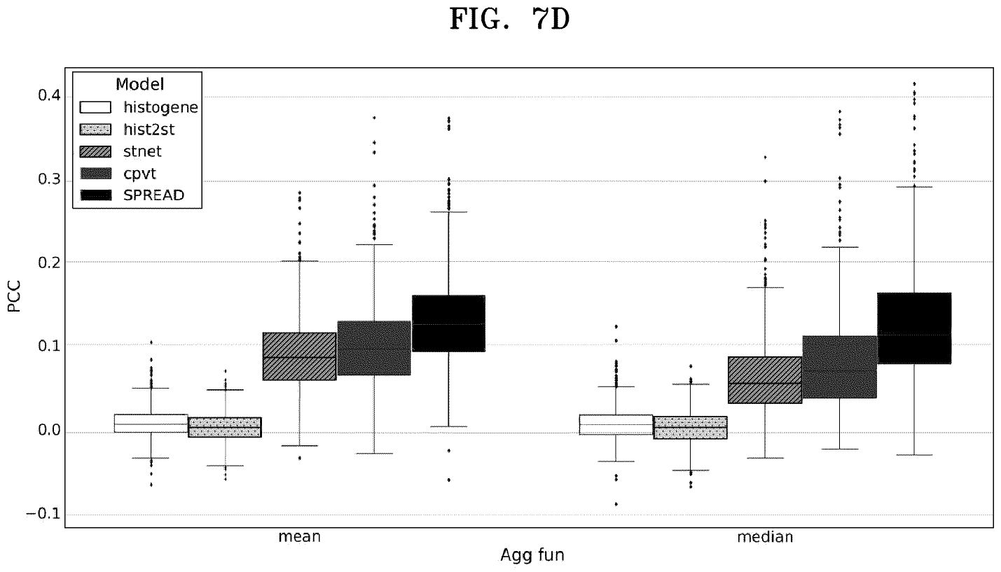

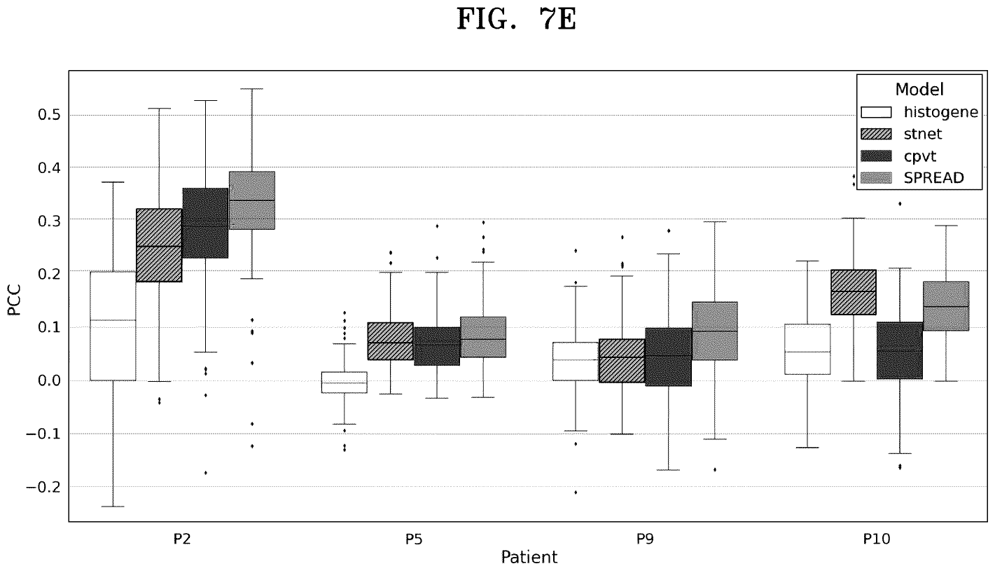

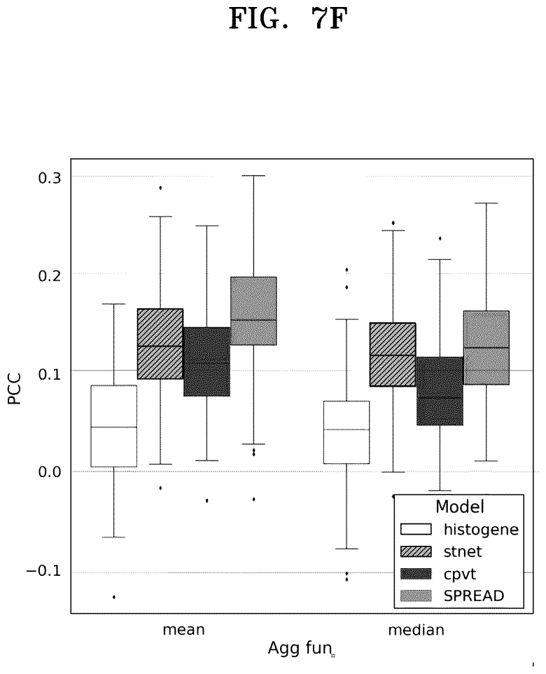

FIGS. 7A to 7F are diagrams quantitatively showing the performance of artificial intelligence models to predict gene expression, according to an embodiment.

FIGS. 7A and 7B show a table for comparing the gene expression prediction performance of a gene expression prediction model (‘SPREAD’) according to an embodiment, with the gene expression prediction performance of gene expression prediction models (‘ST-net’, ‘HistoGene’, ‘Hist2ST’, and ‘CPVT’) according to some embodiments. In FIGS. 7A and 7B, the gene expression prediction performance of each model is the performance of gene expression prediction using the model, and may refer to prediction accuracy, and higher prediction accuracy may indicate higher prediction performance. The prediction accuracy may be calculated/evaluated based on gene expression values predicted by using the model, and ground-truth gene expression values.

In FIGS. 7A and 7B, ‘SPREAD’ may refer to the model illustrated in FIG. 3 (i.e., 314 and 300 of FIG. 3), ‘ST-net’ may refer to the model described in the first prior paper (Abubakar Abid, Alma Andersson, Ake Borg, Jonas Maaskola, Joakim Lundeberg, and James Zou. Integrating spatial gene expression and breast tumor morphology via deep learning. Nature biomedical engineering, 4(8):827-834, 2020.), ‘HistoGene’ may refer to the model described in the second prior paper (Minxing Pang, Kenong Su, and Mingyao Li. Leveraging information in spatial transcriptomics to predict super-resolution gene expression from histology images in tumors. bioRxiv, 2021.), and ‘Hist2ST’ may refer to the model described in the third prior paper (Yuansong Zeng, Zhuoyi Wei, Weijiang Yu, Rui Yin, Yuchen Yuan, Bingling Li, Zhonghui Tang, Yutong Lu, and Yuedong Yang. Spatial transcriptomics pre diction from histology jointly through transformer and graph neural networks. Briefings in Bioinformatics, 23(5):bbac297, 2022.). In FIGS. 7A and 7B, ‘ST-net’, ‘HistoGene’, and ‘Hist2ST’ may be models trained by adopting hyperparameters used in the respective prior papers. In FIGS. 7A and 7B, ‘CPVT’ is a partial modification of an existing CPVT model including one or more encoders and a PEG, and the same hyperparameters as a global embedding model of ‘SPREAD’ may be applied to other component than the PEG. Through the prediction performance of ‘CPVT’, it is possible to compare the performance of ‘SPREAD’, which considers both global information and local information, with a model that performs prediction focusing on only global information (e.g., ‘CPVT’).

FIG. 7A may show a result of a test performed with two public ST datasets including a breast cancer ST dataset (‘ST-Breast’) and a skin cancer ST dataset (′ST-Skin′). FIG. 7B may show a result of a test performed with three external Visium datasets (′10× Visium-1′, ‘10× Visium-2’, and ‘10× Visium-3’) from 10× Genomics (i.e. the next version of ST with a higher resolution).

FIG. 7A shows a result of leave-one-out cross-validation (LOOCV) for each model using a ST dataset, and may include Pearson correlation coefficient (PCC)(A), PCC(M), mean squared error (MSE), and mean absolute error (MAE) values. FIG. 7B shows a result of a generalization performance test for each model using external Visium data, and may include PCC(A), PCC(M), MSE, and MAE values. In FIGS. 7A and 7B, PCC may refer to Pearson correlation coefficient, MSE may refer to mean squared error, MAE may refer to mean absolute error, and PCC, MSE, and MAE may be used as metrics to evaluate the accuracy of regression tasks. PCC(A) and PCC(M) may refer to mean PCC and median PCC, respectively, and ‘-’ may indicate unavailability.

FIG. 7A may include mean PCCs, MSEs, and MAEs from all folds calculated through LOOCV on each patient data using ST data. FIG. 7B may include results of tests performed by using respective Visium data for models trained with breast cancer ST data. As preprocessing of images for a performance test of each model, WSIs may be cropped into patches having a size of 224×224 by using center coordinates. Gene expression values may be normalized by the sum of gene expression values within each point, and log transformation may be applied.

In a model training phase for validating the performance of the ‘SPREAD’ model of FIGS. 7A and 7B, an Adam optimizer with a constant learning rate of 0.0001 may be used to update weights of the model. For self-distillation of the ‘SPREAD’ model, hyperparameters a and y may be set to 0.3 and 0.03, and a global embedding model of the ‘SPREAD’ model may include four encoding layers (i.e. encoders) and one PEGH located after the first encoder.

In a model training phase, the mini-batch size may be set to 256, and in a model testing operation, the mini-batch size may be set to 1. In a training phase, the model may be trained by using patches as much as the mini-batch size among all patches of a training WSI, and initial feature data extracted from each patch of the training WSI, that is, pairs of (patches, initial feature data extracted from each patch of the WSI) as much as the mini-batch size. In a test phase, the model may perform inference by using all patches of the training WSI, and initial feature data extracted from each patch of the training WSI, that is, pairs of (patches, initial features extracted from each patch of the WSI). In addition, a pair of a WSI and a gene expression matrix included in the ST data may be used as a ground-truth label (i.e., a ground-truth gene expression value).

The breast cancer ST dataset is data regarding 7 patients, and may include 4 or 5 replicates for each patient. The skin ST dataset is data regarding 4 patients, may include 3 replicates for each patient. Because the replicates are generated from exactly the same tissue sample, LOOCV may be performed on each patient for accurate evaluation. That is, replicates for one patient may be used for nay one of model training or a test.

In order to determine genes to be predicted for performance evaluation, a filtering process may be performed. In detail, after the top N (e.g., 1000) highly variable genes within each data are selected, genes of which the frequency of expression in all patches is less than a threshold (e.g., 1000) may be excluded. FIG. 7A may show results of evaluation of the prediction performance on expression of 785 genes selected from breast cancer ST data and 171 genes selected from skin cancer ST data through the above-described filtering process. That is, the ‘ST-Breast’ column in FIG. 7A may represent the prediction performance of each model on expression of 785 genes according to LOOCV performed by using breast cancer ST data, and the ‘ST-Skin’ column may represent the prediction performance of each model on expression of 171 genes according to LOOCV performed by using skin cancer ST data.

According to values for evaluation indices on the breast cancer ST dataset and the skin cancer ST dataset in FIG. 7A, the performance of ‘SPREAD’ shows superior performance compared to the performance of other models. In detail, FIG. 7A shows that ‘SPREAD’ achieves a 45.5% increase in mean PCC (′PCC(A)′) and a 2.8% decrease in MSE for the breast cancer ST dataset, compared to ‘ST-net’, which has the best performance among other models. In addition, FIG. 7A shows that ‘SPREAD’ achieves a 13.6% increase in mean PCC and a 9.6% decrease in MSE for the skin cancer dataset compared to ‘ST-net’. In addition, compared to ‘CPVT’, which uses only global information to predict gene expression, ‘SPREAD’ achieves a 30% increase in mean PCC and a 2.5% decrease in MSE for the breast cancer ST dataset. Therefore, FIG. 7A may suggest that ‘SPREAD’ achieves improved prediction performance compared to other models, and that both local information and global context are important in predicting spatial gene expression from a histology image (or a WSI).

FIG. 7B may show results of performance evaluation of each model for independent datasets from various sources. In the performance evaluation of FIG. 7B, three publicly available 10× Genomics Visium data from three different patients may be used to test the model in an independent cohort. For performance evaluation, 10× Genomics Visium data may be preprocessed in the same manner as performed on ST data. For example, patches cropped to a size of 224×224 from a histology image included in 10× Genomics Visium data may be used, and the patches may be pre-embedded for use as input data for the global embedding model. FIG. 7B may show results of performance evaluation on each of three pieces of 10× Genomics Visium data.

FIG. 7B shows results of testing generalization performance on external test data. Models to be tested for performance may be trained based on the optimal number of epochs that is calculated according to the LOOCV results of FIG. 7A, and the breast cancer ST dataset. Each model trained based on the breast cancer ST dataset may be tested by using Visium data. FIG. 7B shows that ‘SPREAD’ achieves the best performance in PCC(A) and PCC(M) compared to ‘ST-net’ and ‘CPVT’. Thus, FIG. 7B may suggest that ‘SPREAD’ is a more robust and generalizable model than the other models. In addition, FIG. 7B shows that ‘CPVT’ achieves the second best performance and the best performance in error metrics (i.e. MSE and MAE). Thus, FIG. 7B may suggest that feature embedding considering a global context is important for robust prediction performance.

Therefore, FIGS. 7A and 7B show that the ‘SPREAD’ model according to an embodiment achieves better performance for the ST dataset than models according to some embodiments, and excellent performance for other external data, and thus is a better generalized model.

FIGS. 7C and 7D shows a graph of performance results on the breast cancer ST dataset of FIG. 7A. FIGS. 7E and 7F shows a graph of performance results on the skin cancer ST dataset of FIG. 7A. The x-axis of FIGS. 7C and 7E may represent patient data used as test data. For example, ‘A’ on the x-axis in FIG. 7C may indicate that each model was trained by using data of patients ‘B, C, D, E, F, and G’, and tested with data of patient ‘A’. Similarly, ‘P2’ on the x-axis of FIG. 7E may indicate that each model was trained by using data of patients ‘P5, P9, and P10’, and tested with data of patient ‘P2’. The y-axis of FIGS. 7C and 7E may represent PCC values for genes to be predicted. The distribution graphs of FIGS. 7C and 7E may represent distributions of PCC values of each model for a plurality of genes.

FIGS. 7D and 7F may show a distribution of means and a distribution of median values of PCC values in a plurality of folds for each gene. For example, the distribution graph of FIG. 7D may include the mean of PCC values for a first gene in seven folds, as a mean PCC of the first gene, and a median PCC value for the first gene in the seven folds, as a median PCC of the first gene. Similarly, the distribution graph of FIG. 7F may include the mean of PCC values for the first gene in four folds, as a mean PCC of the first gene, and a median PCC value for the first gene in the four folds, as a median PCC of the first gene. FIGS. 7D and 7F may show that the “SPREAD” model according to an embodiment has higher distributions of mean PCCs and median PCCs compared to models according to some embodiments.

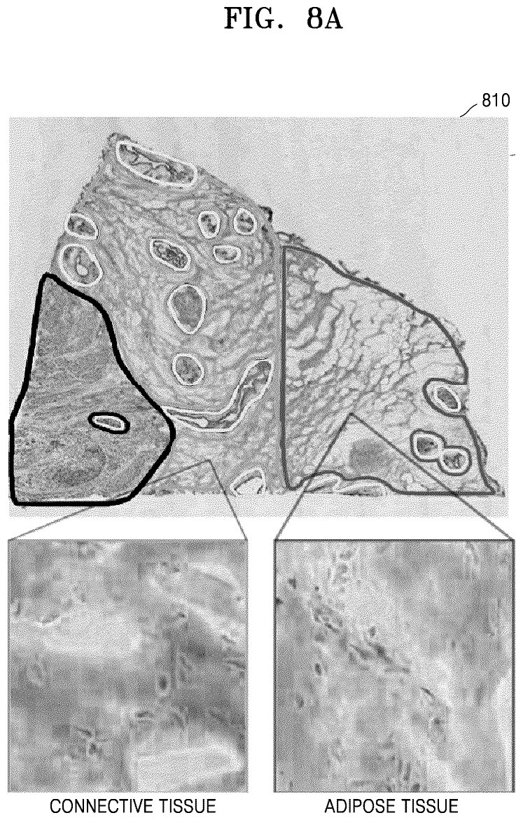

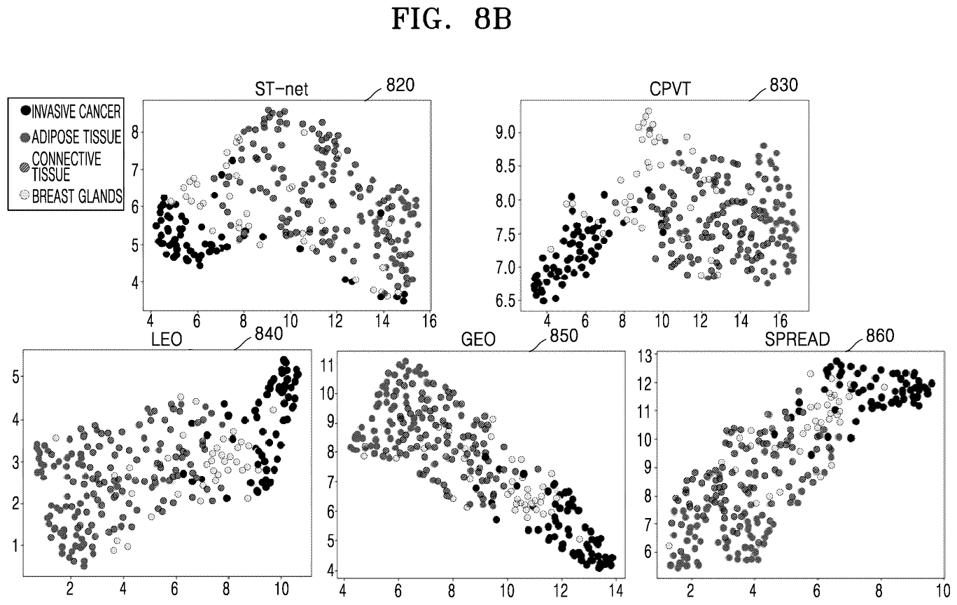

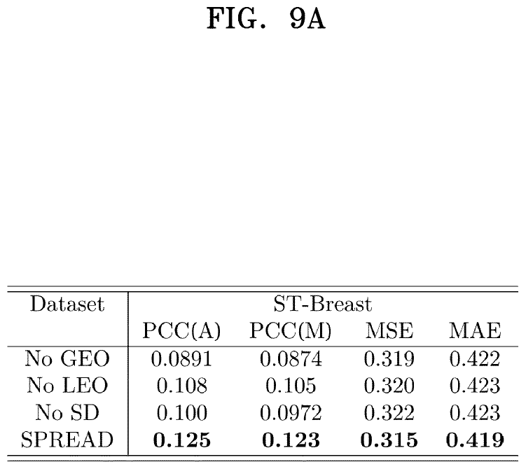

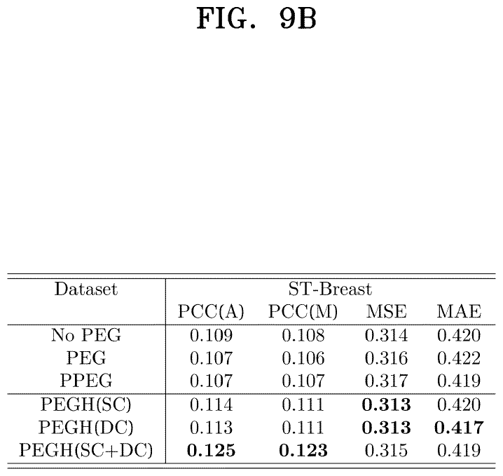

The settings for performance evaluation described above with reference to FIGS. 7A to 7F may also be applied to FIGS. 8A, 8B, 9A, and 9B.

FIGS. 8A and 8B are diagrams qualitatively showing the prediction performance of an artificial intelligence model according to an embodiment.

By using tissue type annotations in the breast cancer ST data, an indirect and qualitative evaluation of the performance of the gene expression prediction model (‘SPREAD’) according to an embodiment may be performed. Because gene expression significantly varies depending on tissue type, it may be assumed that a model (or a method) with good gene expression prediction performance is able to distinguish between different tissue types, even without label information.

Graphs 820, 830, 840, 850, 860 of FIG. 8B may be visualizations of latent feature maps (e.g. feature maps input to FC layers and intermediate generated feature maps) of ‘SPREAD’ and other models (′ST-net′, ‘CPVT’, ‘LEO’, and ‘GEO’) for a histology image 810 of FIG. 8A. In detail, FIG. 8B may show results of extracting feature maps (i.e., latent feature maps) from all patches within the histology image 810 of FIG. 8A by using the respective models, and projecting the extracted feature maps into a two-dimensional space by using the Uniform Manifold Approximation and Projection (UMAP) algorithm. ‘SPREAD’ may represent the model illustrated in FIG. 3 (e.g., 300 of FIG. 3), ‘ST-net’ may represent the model described in the first prior paper (Abubakar Abid, Alma Andersson, Ake Borg, Jonas Maaskola, Joakim Lundeberg, and James Zou. Integrating spatial gene expression and breast tumour morphology via deep learning. Nature biomedical engineering, 4(8):827-834, 2020.), ‘LEO’ may represent the local embedding model illustrated in FIG. 3 (i.e., 330 of FIG. 3), and ‘GEO’ may represent the global embedding model illustrated in FIG. 3 (i.e., 320 of FIG. 3).

In the graphs 820, 830, 840, 850, and 860 of FIG. 8B, feature data for each patch (i.e., a feature map for each patch extracted by the model) may be projected into a two-dimensional space, and thus indicated as a dot corresponding to each patch. The shading of the dot may indicate the tissue type (e.g., the main tissue type) of the patch at the corresponding position among four tissue types (invasive cancer, connective tissue, breast glands, and adipose tissue). Referring to FIG. 8B, in the graph 860 of ‘SPREAD’, dots corresponding to the same tissue type are distributed at similar positions, and dots corresponding to different tissue types are distributed to be distinguished from each other. Thus, the graphs 820, 830, 840, 850, and 860 of FIG. 8B may indicate that ‘SPREAD’ better distinguishes between the four tissue types compared to the other models.

Among the four tissue types, connective tissue and adipose tissue are almost indistinguishable when focusing on a local region, and may be clearly distinguished only when compared over the entire image at a global scale. The graph 860 of ‘SPREAD’ in FIG. 8B shows that ‘SPREAD’ distinguishes well between patches of connective tissue and adipose tissue. In addition, according to the graphs 820, 830, 840, 850, and 860 of FIG. 8B, ‘STnet’ and ‘LEO’, which are models that rely on local features of patches, tend to show poorer performance in distinguishing between connective tissue and adipose tissue compared to ‘CPVT’, ‘GEO’, and ‘SPREAD’, which are models that consider a global context. Thus, FIG. 8B may indirectly show that it is important to consider a global context along with local features when predicting spatial gene expression.

FIGS. 9A and 9B are diagrams showing a performance contribution of each component of an artificial intelligence model according to an embodiment.

FIGS. 9A and 9B may show a contribution of each component (or each component model) of a gene expression prediction model (‘SREAD’) according to an embodiment, through an ablation study. ‘SPREAD’ may refer to the model illustrated in FIG. 3 (e.g., 300 of FIG. 3). The gene expression prediction performance of the model may refer to the performance of gene expression prediction using the model. PCC(A), PCC(M), MSE, and MAE of FIGS. 9A and 9B are predictive performance indicators, and high values of PCC(A) and PCC(M) and low values of MSE and MAE may indicate high performance.

FIGS. 9A and 9B may show a contribution of each component of the ‘SPREAD’ model according to an embodiment, through a performance test using the breast cancer ST dataset. In detail. FIG. 9A may show the prediction performance of a model without each component. Components of SPREAD excluded in FIG. 9A may include a global embedding model (‘GEO’), a local embedding model (‘LEO’), and a self-distillation scheme (‘SD’), which is a learning method component of the SPREAD model. The self-distillation scheme may be applied in the training phase and also in the inference phase to calculate a final gene expression prediction value. In FIG. 9A, ‘No GEO’ may represent a model obtained by removing the global embedding model from ‘SPREAD’, ‘No LEO’ may represent a model obtained by removing the local embedding model and the self-distillation scheme from ‘SPREAD’, and ‘No SD’ may represent a model obtained by removing the self-distillation scheme from ‘SPREAD’.

Referring to FIG. 9A, the prediction performance degrades in a case in which the self-distillation scheme is not applied. These results may indicate that the self-distillation scheme according to an embodiment significantly contributes to integration of local-global information and prediction of gene expression using dual embedded features. In addition, the performance degradation is greatest in the model without the global embedding model (‘No GEO’). In addition, because the SPREAD model has the highest PCC value and the lowest MSE value, FIG. 9A may indicate that the SPREAD model has the best performance.

FIG. 9B may show performance differences according to each positional encoding method. For example, FIG. 9B shows mean PCCs, median PCCs, MSEs, and MAEs for a case in which no positional encoding is performed (‘No PEG’), a case in which positional encoding is performed by using a PEG method (‘PEG’), a case in which positional encoding is performed by using a PPEG method (‘PPEG’), a case in which positional encoding is performed by using a PEGH method using only a standard 3×3 convolution kernel (′PEGH(SC)′), a case in which positional encoding is performed by using a PEGH method using only a deformable 3×3 convolution kernel (′PEGH(DC)′), and a case in which positional encoding is performed by using a PEGH method using both a standard 3×3 convolution kernel and a deformable 3×3 convolution kernel (′PEGH(SC+DC)′).

Referring to FIG. 9B, the prediction performance of models applying PEGH (′PEGH(SC)′, ‘PEGH(DC)’, and ‘PEGH(SC+DC)’ of FIG. 9B) is superior to the prediction performance of a model that does not perform positional encoding and a model that performs positional encoding by using the PEG or PPEG method. In addition, the performance of the model that does not perform positional encoding is similar to, or rather superior to, the prediction performance of the model that performs positional encoding by using the PEG or PPEG method. It may be difficult to capture spatial information between patches by simply performing a convolution operation.

Referring to FIG. 9B, the model that performs positional encoding by using the PEGH method using both the standard 3×3 convolution kernel and the deformable 3×3 convolution kernel has the highest PCC value. Furthermore, the model that performs positional encoding by using the PEGH method using only the deformable 3×3 convolution kernel has the lowest MSE and MAE values. Thus, in processing a WSI having an arbitrary shape, a positional encoding method using a deformable convolution kernel may improve the gene expression prediction performance of the model.

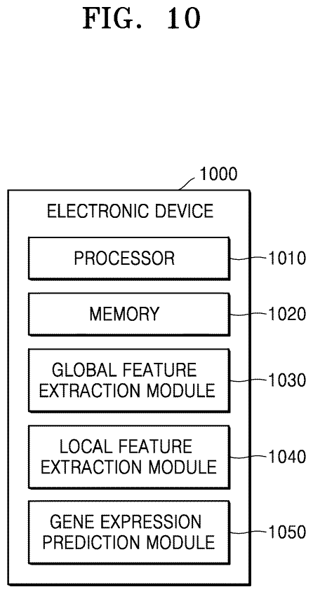

FIG. 10 is a block diagram illustrating an example of an electronic device according to an embodiment.

An electronic device 1000 illustrated in FIG. 10 is a device for predicting patch-level gene expression from a histology image by using an artificial intelligence model, and may be a user terminal or a server device. For example, the electronic device 1000 may be a user terminal such as a desktop computer, a laptop computer, a notebook, a smart phone, a tablet personal computer (PC), a mobile phone, a smart watch, a wearable device, an augmented reality (AR) device, or a virtual reality (VR) device.

In an embodiment, an electronic device for training/updating an artificial intelligence model to predict patch-level gene expression from a histology image may be a user terminal or a server device that is the same as or different from the electronic device 1000 for performing prediction/inference by using an artificial intelligence model. For example, in a case in which the electronic device for training/updating an artificial intelligence model and the electronic device 1000 (i.e., the electronic device for performing an inference operation) are different from each other, the electronic device 1000 may receive a trained/updated artificial intelligence model from the electronic device for training/updating an artificial intelligence model. One or more embodiments to be described below regarding the electronic device 1000 may also be applied to the electronic device for training/updating an artificial intelligence model to predict patch-level gene expression from a histology image.

In an embodiment, the artificial intelligence model may be updated as an inference operation is performed. For example, in a case in which the electronic device for updating an artificial intelligence model and the electronic device 1000 are the same as each other, the electronic device 1000 may perform prediction/inference by using an artificial intelligence model and update the artificial intelligence model at the same time. For example, in a case in which the electronic device for updating an artificial intelligence model and the electronic device 1000 are different from each other, the electronic device for updating an artificial intelligence model may perform prediction/inference by using an artificial intelligence model, receive identified/generated/calculated data, and update the artificial intelligence model based on the received data.

In an embodiment, the electronic device 1000 may include at least one processor 1010 and a memory 1020, but is not limited thereto. The processor 1010 may be electrically connected to the components included in the electronic device 1000 to perform computations or data processing for control and/or communication of the components included in the electronic device 1000. In an embodiment, the processor 1010 may load, into the memory, a request, a command, or data received from at least one of other components, process the request, command, or data, and store process result data in the memory. According to various embodiments, the processor 1010 may include at least one of general-purpose processors such as a central processing unit (CPU), an application processor (AP), or a digital signal processor (DSP), dedicated graphics processors such as a graphics processing unit (GPU) or a vision processing unit (VPU), or dedicated artificial intelligence processors such as a neural processing unit (NPU).

The processor 1010 may perform control to process input data according to predefined operation rules or an artificial intelligence model (e.g., a neural network model) stored in the memory 1020. In a case in which the processor 1010 is a dedicated artificial intelligence processor, the dedicated artificial intelligence processor may be designed with a hardware structure specialized for processing a particular artificial intelligence model.

The predefined operation rules or artificial intelligence model may be generated via a training process. Here, being generated via a training process may mean that predefined operation rules or artificial intelligence model set to perform desired characteristics (or purposes), is generated by training a basic artificial intelligence model by using a learning algorithm that utilizes a large amount of training data. The training process may be performed by a device itself on which artificial intelligence according to the disclosure is performed, or by a separate server and/or system. Examples of learning algorithms may include, for example, supervised learning, unsupervised learning, semi-supervised learning, and reinforcement learning, but are not limited thereto.

The memory 1020 may be electrically connected to the processor 1010, and may store one or more modules related to the operations of the components included in the electronic device 1000, at least one learning model, a program, instructions, or data. For example, the memory 1020 may store one or more modules, learning models, programs, instructions, or data for the processor 1010 to perform processing and control. The memory 1020 may include at least one of a flash memory-type storage medium, a hard disk-type storage medium, a multimedia card micro-type storage medium, card-type memory (e.g., SD or XD memory), random-access memory (RAM), static RAM (SRAM), read-only memory (ROM), electrically erasable programmable ROM (EEPROM), programmable ROM (PROM), magnetic memory, a magnetic disc, and an optical disc.

In an embodiment, the memory 1020 may store data and information received or generated by the electronic device 1000. For example, the memory 1020 may store a received histology image, weight values of an artificial intelligence model, and the like. For example, the memory 1020 may store a gene expression prediction result for a histology image (or a patch). For example, the memory 1020 may store data and information received or generated by the electronic device 1000 in a compressed form.

The module or model included in the memory 1020 may be executed under control of or according to a command of the processor 1010, and may include a program, a model, or an algorithm configured to perform operations of deriving output data for input data. The memory 1020 may include at least one neural network model, an artificial intelligence model, a machine learning model, a statistical model, an algorithm, and the like for image processing. In an embodiment, the memory 1020 may include an artificial intelligence model configured to predict/infer patch-level gene expression from a histology image. The memory 1020 may include a plurality of parameter values (weight values) constituting an artificial intelligence model.

The artificial intelligence model may include a plurality of neural network layers. Each of the neural network layers may have a plurality of weight values, and may perform a neural network arithmetic operation via an arithmetic operation between an arithmetic operation result of a previous layer and the plurality of weight values. The plurality of weight values in each of the neural network layers may be optimized by a result of training the artificial intelligence model. For example, the plurality of weight values may be updated to reduce or minimize a loss or cost value obtained by the artificial intelligence model during a training process. The artificial intelligence model may include a deep neural network (DNN), and may be, for example, a CNN, a long short-term memory (LSTM), a DNN, a recurrent neural network (RNN), a restricted Boltzmann machine (RBM), a deep belief network (DBN), a bidirectional recurrent deep neural network (BRDNN), a transformer, a deep Q-network, or the like, but is not limited thereto. The artificial intelligence model may include a statistical method model, for example, logistic regression, a Gaussian Mixture Model (GMM), a support vector machine (SVM), latent Dirichlet allocation (LDA), or decision tree, but is not limited thereto.

The artificial intelligence model may include one or more sub-models. For example, the artificial intelligence model may include sub-models that may be distinguished from each other by function, output, or structure. In an embodiment, a model configured to predict gene expression from a histology image may include an initial feature extraction model, a global feature extraction model (i.e., a global embedding model), a local feature extraction model (i.e., a local embedding model), and a combined prediction model. The global feature extraction model may include one or more encoders (or encoding models) and a positional information encoder (or a positional information encoding model). For example, at least one sub-model is an independent artificial intelligence model and may be individually trained or perform inference. For example, at least one sub-model may be trained or perform inference together with other sub-models constituting the artificial intelligence model.

Some modules that perform at least one operation of the electronic device 1000 may be implemented as hardware modules, software modules, and/or a combination thereof. The memory 1020 may include software modules that perform at least some of the operations of the electronic device 1000 described above. In an embodiment, a module included in the memory 1020 may be executed by the processor 1010 to perform an operation. For example, a model included in memory 1020 may constitute a module, be included in a module, be executed by a module, or be a module itself. At least some of the modules of the electronic device 1000 may include a plurality of sub-modules or may constitute one module.

Referring to FIG. 10, the electronic device 1000 may include, but is not limited to, a global feature extraction module 1030, a local feature extraction module 1040, and a gene expression prediction module 1050. For example, the electronic device 1000 may include only some of the global feature extraction module 1030, the local feature extraction module 1040, and the gene expression prediction module 1050. In this case, the other modules may be included in at least one other electronic device, and the electronic device 1000 and the other electronic device may be connected to each other or communicate with each other in a wired or wireless manner to constitute a system.

FIG. 10 illustrates that the global feature extraction module 1030, local feature extraction module 1040, and gene expression prediction module 1050 are separate components from the processor 1010 and the memory 1020, but the disclosure is not limited thereto. For example, at least one of the global feature extraction module 1030, the local feature extraction module 1040, or the gene expression prediction module 1050 may be included in the processor 1010 as a hardware module, or may be a separate hardware model. For example, at least one of the global feature extraction module 1030, the local feature extraction module 1040, or the gene expression prediction module 1050 may be included in the memory 1020 as a software module. For example, a software component of each module may be included in memory 1020.

In an embodiment, the global feature extraction module 1030 may include a model configured to extract patch-level global features from a histology image (i.e., a global feature extraction model, a global embedding model, or a first artificial intelligence model). In an embodiment, the local feature extraction module 1040 may include a model configured to extract patch-level local features from a histology image (i.e., a local feature extraction model, a local embedding model, or a second artificial intelligence model). In an embodiment, the gene expression prediction module 1050 may include a model configured to predict patch-level gene expression based on patch-level global features and local features of a histology image (e.g., a combined prediction model, a predictor, or a third artificial intelligence model).

The electronic device 1000 may include more components than those illustrated in FIG. 10. For example, the electronic device 1000 may further include a communication interface (or a communication module) for communication with an external device. For example, the electronic device 1000 may further include an input/output device and/or an input/output interface. For example, the electronic device 1000 may further include a camera sensor for generating a histology image.

The descriptions provided above with reference to FIGS. 1A to 10 may have been omitted, and the embodiments described above with reference to FIGS. 1A to 10 may be applied/implemented in combination with each other.

The at least one processor 1010 may control a series of processes such that the electronic device 1000 may operate according to at least one of the embodiments described above with reference to FIGS. 1A to 10. That is, the at least one processor 1010 may control each component of the electronic device 1000 to operate. In an embodiment, the at least one processor 1010 may execute one or more instructions stored in the memory. In an embodiment, the at least one processor 1010 may execute the one or more instructions to perform an operation of predicting patch-level gene expression from a histology image by using an artificial intelligence model.

In an embodiment, the at least one processor may identify a first patch of the histology image divided into a plurality of patches, initial feature data of the first patch, and initial feature data of a second patch of the histology image. In an embodiment, the at least one processor may extract global feature data of the first patch based on the initial feature data of the first patch and the initial feature data of the second patch, by using a first artificial intelligence model. In an embodiment, the at least one processor may extract local feature data of the first patch from the first patch by using a second artificial intelligence model. In an embodiment, the at least one processor may predict a gene expression value for the first patch based on the global feature data of the first patch and the local feature data of the first patch, by using a third artificial intelligence model.

In an embodiment, the at least one processor may extract the initial feature data of the first patch from the first patch, and extract the initial feature data of the second patch from the second patch, by using a pre-trained artificial intelligence model.

In an embodiment, the at least one processor may generate local-global feature data of the first patch by concatenating the global feature data of the first patch with the local feature data of the first patch. In an embodiment, the at least one processor may predict the gene expression value for the first patch based on the local-global feature data of the first patch, by using the third artificial intelligence model.

In an embodiment, the at least one processor may extract first local feature data and second local feature data of the first patch from the first patch, by using the second artificial intelligence model. In an embodiment, the at least one processor may generate first local-global feature data in which the first local feature data of the first patch is concatenated with the global feature data of the first patch, and second local-global feature data in which the second local feature data of the first patch is concatenated with the global feature data of the first patch. In an embodiment, the at least one processor may predict a first gene expression value for the first patch based on the first local-global feature data, and predict a second gene expression value for the first patch based on the second local-global feature data, by using the third artificial intelligence model. In an embodiment, the at least one processor may determine a final gene expression value for the first patch based on at least one of the first gene expression value or the second gene expression value.

In an embodiment, the second artificial intelligence model may include a plurality of sequentially connected layers. In an embodiment, the first local feature data may be output data from a deepest layer among the plurality of layers. In an embodiment, the second local feature data may be data generated based on output data from a layer other than the deepest layer among the plurality of layers.

In an embodiment, the first artificial intelligence model may include a positional information encoder configured to encode patch positional information in the histology image. In an embodiment, the at least one processor may extract the global feature data of the first patch in which positional information of the first patch is encoded, by using the positional information encoder.

In an embodiment, the at least one processor may perform at least one self-attention operation based on the initial feature data of the first patch and the initial feature data of the second patch. In an embodiment, the at least one processor may perform a convolution operation on result data of the at least one self-attention operation, based on the positional information of the first patch and positional information of the second patch. In an embodiment, the at least one processor may perform a deformable convolution operation on the result data of the at least one self-attention operation, based on the positional information of the first patch and the positional information of the second patch. In an embodiment, the at least one processor may extract the global feature data of the first patch in which the positional information of the first patch is encoded, based on result data of the convolution operation and result data of the deformable convolution operation.

A method of predicting patch-level gene expression from a histology image by using an artificial intelligence model according to an embodiment may be performed by an electronic device. In an embodiment, the method may include identifying a first patch of the histology image divided into a plurality of patches, initial feature data of the first patch, and initial feature data of a second patch of the histology image. In an embodiment, the method may include extracting global feature data of the first patch based on the initial feature data of the first patch and the initial feature data of the second patch, by using a first artificial intelligence model. In an embodiment, the method may include extracting local feature data of the first patch from the first patch by using a second artificial intelligence model. In an embodiment, the method may include predicting a gene expression value for the first patch based on the global feature data of the first patch and the local feature data of the first patch, by using a third artificial intelligence model.

In an embodiment, the identifying of the first patch of the histology image divided into the plurality of patches, the initial feature data of the first patch, and the initial feature data of the second patch of the histology image may include extracting the initial feature data of the first patch from the first patch, and the initial feature data of the second patch from the second patch, by using a pre-trained artificial intelligence model.

In an embodiment, the predicting of the gene expression value for the first patch may include generating local-global feature data of the first patch by concatenating the global feature data of the first patch with the local feature data of the first patch. In an embodiment, the predicting of the gene expression value for the first patch may include predicting the gene expression value for the first patch based on the local-global feature data of the first patch, by using the third artificial intelligence model.

In an embodiment, the extracting of the local feature data of the first patch from the first patch by using the second artificial intelligence model may include extracting first local feature data and second local feature data of the first patch from the first patch, by using the second artificial intelligence model. In an embodiment, the predicting of the gene expression value for the first patch may include generating first local-global feature data in which the first local feature data of the first patch is concatenated with the global feature data of the first patch, and second local-global feature data in which the second local feature data of the first patch is concatenated with the global feature data of the first patch. In an embodiment, the predicting of the gene expression value for the first patch may include predicting a first gene expression value for the first patch based on the first local-global feature data, and predicting a second gene expression value for the first patch based on the second local-global feature data, by using the third artificial intelligence model. In an embodiment, the predicting of the gene expression value for the first patch may include determining a final gene expression value for the first patch based on at least one of the first gene expression value or the second gene expression value.

In an embodiment, the second artificial intelligence model may include a plurality of sequentially connected layers. In an embodiment, the first local feature data may be output data from a deepest layer among the plurality of layers. In an embodiment, the second local feature data may be data generated based on output data from a layer other than the deepest layer among the plurality of layers.

In an embodiment, the first artificial intelligence model may include a positional information encoder configured to encode patch positional information in the histology image. In an embodiment, the extracting of the global feature data of the first patch based on the initial feature data of the first patch and the initial feature data of the second patch, by using the first artificial intelligence model may include extracting the global feature data of the first patch in which positional information of the first patch is encoded, by using the positional information encoder.

In an embodiment, the extracting of the global feature data of the first patch in which the positional information of the first patch is encoded, by using the positional information encoder may include performing at least one self-attention operation based on the initial feature data of the first patch and the initial feature data of the second patch. In an embodiment, the extracting of the global feature data of the first patch in which the positional information of the first patch is encoded, by using the positional information encoder may include performing a convolution operation on result data of the at least one self-attention operation, based on the positional information of the first patch and positional information of the second patch. In an embodiment, the extracting of the global feature data of the first patch in which the positional information of the first patch is encoded, by using the positional information encoder may include performing a deformable convolution operation on the result data of the at least one self-attention operation, based on the positional information of the first patch and the positional information of the second patch. In an embodiment, the extracting of the global feature data of the first patch in which the positional information of the first patch is encoded, by using the positional information encoder may include extracting the global feature data of the first patch in which the positional information of the first patch is encoded, based on result data of the convolution operation and result data of the deformable convolution operation.

In an embodiment, the first artificial intelligence model, the second artificial intelligence model, and the third artificial intelligence model may be connected to each other in an end-to-end manner, and trained simultaneously.

In an embodiment, the first artificial intelligence model, the second artificial intelligence model, and the third artificial intelligence model may be trained by using a loss function based on a difference between a gene expression value predicted for a target patch included in a training image, and a ground-truth gene expression value for the target patch.

In an embodiment, the first artificial intelligence model, the second artificial intelligence model, and the third artificial intelligence model may be trained by using a loss function based on a difference between first local-global feature data for a target patch included in a training image, and second local-global feature data for the target patch. In an embodiment, the first local-global feature data for the target patch may be generated by concatenating first local feature data of the target patch, which is output data from a deepest layer of the second artificial intelligence model for the target patch, with global feature data of the target patch. In an embodiment, the second local-global feature data for the target patch may be generated by concatenating second local feature data of the target patch based on output data from a layer other than the deepest layer of the second artificial intelligence model for the target patch, with the global feature data of the target patch.

In an embodiment, the first artificial intelligence model, the second artificial intelligence model, and the third artificial intelligence model may be trained by using a loss function based on a difference between a first gene expression value for a target patch included in a training image, and a second gene expression value for the target patch. In an embodiment, the first gene expression value for the target patch may be predicted based on output data from a deepest layer of the second artificial intelligence model for the target patch. In an embodiment, the second gene expression value for the target patch may be predicted based on output data from a layer other than the deepest layer of the second artificial intelligence model for the target patch.

A program for executing, on a computer, a method of predicting patch-level gene expression from a histology image by using an artificial intelligence model according to an embodiment may be recorded on a computer-readable recording medium.

A machine-readable storage medium may be provided in the form of a non-transitory storage medium. Here, the term ‘non-transitory storage medium’ refers to a tangible device and does not include a signal (e.g., an electromagnetic wave), and the term ‘non-transitory storage medium’ does not distinguish between a case where data is stored in a storage medium semi-permanently and a case where data is stored temporarily. For example, the ‘non-transitory storage medium’ may include a buffer in which data is temporarily stored.

According to an embodiment, methods according to various embodiments disclosed herein may be included in a computer program product and then provided. The computer program product may be traded as commodities between sellers and buyers. The computer program product may be distributed in the form of a machine-readable storage medium (e.g., a compact disc ROM (CD-ROM)), or may be distributed online (e.g., downloaded or uploaded) through an application store or directly between two user devices (e.g., smart phones). In a case of online distribution, at least a portion of the computer program product (e.g., a downloadable app) may be temporarily stored in a machine-readable storage medium such as a manufacturer's server, an application store's server, or a memory of a relay server.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method, performed by an electronic device, of predicting gene expression from a histology image by using an artificial intelligence model, the method comprising:
identifying a first patch of the histology image divided into a plurality of patches, initial feature data of the first patch, and initial feature data of a second patch of the histology image;
extracting global feature data of the first patch based on the initial feature data of the first patch and the initial feature data of the second patch, by using a first artificial intelligence model;
extracting local feature data of the first patch from the first patch by using a second artificial intelligence model; and
predicting a gene expression value for the first patch based on the global feature data of the first patch and the local feature data of the first patch, by using a third artificial intelligence model.

2. The method of claim 1, wherein the identifying of the first patch of the histology image divided into the plurality of patches, the initial feature data of the first patch, and the initial feature data of the second patch of the histology image comprises extracting the initial feature data of the first patch from the first patch, and extracting the initial feature data of the second patch from the second patch, by using a pre-trained artificial intelligence model.

3. The method of claim 1, wherein the predicting of the gene expression value for the first patch comprises:
generating local-global feature data of the first patch by concatenating the global feature data of the first patch with the local feature data of the first patch; and
predicting the gene expression value for the first patch based on the local-global feature data of the first patch, by using the third artificial intelligence model.

4. The method of claim 1, wherein the extracting of the local feature data of the first patch from the first patch by using the second artificial intelligence model comprises extracting first local feature data and second local feature data of the first patch from the first patch, by using the second artificial intelligence model, and
the predicting of the gene expression value for the first patch comprises:
generating first local-global feature data in which the first local feature data of the first patch is concatenated with the global feature data of the first patch, and second local-global feature data in which the second local feature data of the first patch is concatenated with the global feature data of the first patch;
predicting a first gene expression value for the first patch based on the first local-global feature data, and predicting a second gene expression value for the first patch based on the second local-global feature data, by using the third artificial intelligence model; and
determining a final gene expression value for the first patch based on at least one of the first gene expression value or the second gene expression value.

5. The method of claim 4, wherein the second artificial intelligence model comprises a plurality of sequentially connected layers,
the first local feature data is output data from a deepest layer among the plurality of layers, and
the second local feature data is data generated based on output data from a layer other than the deepest layer among the plurality of layers.

6. The method of claim 1, wherein the first artificial intelligence model comprises a positional information encoder configured to encode patch positional information in the histology image, and
the extracting of the global feature data of the first patch based on the initial feature data of the first patch and the initial feature data of the second patch, by using the first artificial intelligence model comprises extracting the global feature data of the first patch in which positional information of the first patch is encoded, by using the positional information encoder.

7. The method of claim 6, wherein the extracting of the global feature data of the first patch in which the positional information of the first patch is encoded, by using the positional information encoder comprises:
performing at least one self-attention operation based on the initial feature data of the first patch and the initial feature data of the second patch;
performing a convolution operation on result data of the at least one self-attention operation, based on the positional information of the first patch and positional information of the second patch;
performing a deformable convolution operation on the result data of the at least one self-attention operation, based on the positional information of the first patch and the positional information of the second patch; and
extracting the global feature data of the first patch in which the positional information of the first patch is encoded, based on result data of the convolution operation and result data of the deformable convolution operation.

8. The method of claim 1, wherein the first artificial intelligence model, the second artificial intelligence model, and the third artificial intelligence model are connected to each other in an end-to-end manner, and trained simultaneously.

9. The method of claim 1, wherein the first artificial intelligence model, the second artificial intelligence model, and the third artificial intelligence model are trained by using a loss function based on a difference between a gene expression value predicted for a target patch included in a training image, and a ground-truth gene expression value for the target patch.

10. The method of claim 1, wherein the first artificial intelligence model, the second artificial intelligence model, and the third artificial intelligence model are trained by using a loss function based on a difference between first local-global feature data for a target patch included in a training image, and second local-global feature data for the target patch,
the first local-global feature data for the target patch is generated by concatenating first local feature data of the target patch, which is output data from a deepest layer of the second artificial intelligence model for the target patch, with global feature data of the target patch, and
the second local-global feature data for the target patch is generated by concatenating second local feature data of the target patch based on output data from a layer other than the deepest layer of the second artificial intelligence model for the target patch, with the global feature data of the target patch.

11. The method of claim 1, wherein the first artificial intelligence model, the second artificial intelligence model, and the third artificial intelligence model are trained by using a loss function based on a difference between a first gene expression value for a target patch included in a training image, and a second gene expression value for the target patch,
the first gene expression value for the target patch is predicted based on output data from a deepest layer of the second artificial intelligence model for the target patch, and
the second gene expression value for the target patch is predicted based on output data from a layer other than the deepest layer of the second artificial intelligence model for the target patch.

12. A non-transitory computer-readable recording medium having recorded thereon a program for executing, on a computer, the method of claim 1.

13. An electronic device for predicting gene expression from a histology image by using an artificial intelligence model, the electronic device comprising:
a memory storing one or more instructions; and
at least one processor configured to identify a first patch of the histology image divided into a plurality of patches, initial feature data of the first patch, and initial feature data of a second patch of the histology image, extract global feature data of the first patch based on the initial feature data of the first patch and the initial feature data of the second patch, by using a first artificial intelligence model, extract local feature data of the first patch from the first patch by using a second artificial intelligence model, and predict a gene expression value for the first patch based on the global feature data of the first patch and the local feature data of the first patch, by using a third artificial intelligence model.

14. The electronic device of claim 13, wherein the at least one processor is further configured to extract the initial feature data of the first patch from the first patch, and extract the initial feature data of the second patch from the second patch, by using a pre-trained artificial intelligence model.

15. The electronic device of claim 13, wherein the at least one processor is further configured to generate local-global feature data of the first patch by concatenating the global feature data of the first patch with the local feature data of the first patch, and predict the gene expression value for the first patch based on the local-global feature data of the first patch, by using the third artificial intelligence model.

16. The electronic device of claim 13, wherein the at least one processor is further configured to extract first local feature data and second local feature data of the first patch from the first patch, by using the second artificial intelligence model, generate first local-global feature data in which the first local feature data of the first patch is concatenated with the global feature data of the first patch, and second local-global feature data in which the second local feature data of the first patch is concatenated with the global feature data of the first patch, predict a first gene expression value for the first patch based on the first local-global feature data, and predict a second gene expression value for the first patch based on the second local-global feature data, by using the third artificial intelligence model, and determine a final gene expression value for the first patch based on at least one of the first gene expression value or the second gene expression value.

17. The electronic device of claim 16, wherein the second artificial intelligence model comprises a plurality of sequentially connected layers,
the first local feature data is output data from a deepest layer among the plurality of layers, and
the second local feature data is data generated based on output data from a layer other than the deepest layer among the plurality of layers.

18. The electronic device of claim 13, wherein the first artificial intelligence model comprises a positional information encoder configured to encode patch positional information in the histology image, and
the at least one processor is further configured to extract the global feature data of the first patch in which positional information of the first patch is encoded, by using the positional information encoder.

19. The electronic device of claim 18, wherein the at least one processor is further configured to perform at least one self-attention operation based on the initial feature data of the first patch and the initial feature data of the second patch, perform a convolution operation on result data of the at least one self-attention operation, based on the positional information of the first patch and positional information of the second patch, perform a deformable convolution operation on the result data of the at least one self-attention operation, based on the positional information of the first patch and the positional information of the second patch, and extract the global feature data of the first patch in which the positional information of the first patch is encoded, based on result data of the convolution operation and result data of the deformable convolution operation.

20. The electronic device of claim 13, wherein
the first artificial intelligence model, the second artificial intelligence model, and the third artificial intelligence model are trained by using a loss function based on a difference between a first gene expression value for a target patch included in a training image, and a second gene expression value for the target patch,
the first gene expression value for the target patch is predicted based on output data from a deepest layer of the second artificial intelligence model for the target patch, and
the second gene expression value for the target patch is predicted based on output data from a layer other than the deepest layer of the second artificial intelligence model for the target patch.
